(19) United States
(12) Patent Application Publication
(10) Pub. No.: US 2026/0235604 A1
(43) Pub. Date: Aug. 13, 2026

(54) A NOVEL MARKER FOR CARDIOMYOCYTES DEDIFFERENTIATION

(71) Applicant: National University of Singapore, Singapore (SG)

(72) Inventors: Yike ZHU, Singapore (SG); Roger SY FOO, Singapore (SG)

(21) Appl. No.: 19/471,349

(22) PCT Filed: Mar. 15, 2024
(86) PCT No.: PCT/SG2024/050156

§ 371 Date: Sep. 30, 2025

(30) Foreign Application Priority Data
Mar. 30, 2023 (SG) 10202300882X

(51) Int. Cl. G01N 33/573; C12N 5/077; C12N 15/113; C12N 15/85; C12Q 1/25; C12Q 1/6809; C12Q 1/6883; G01N 33/50

(52) U.S. Cl. CPC G01N 33/573; C12N 5/0657; C12N 15/1137; C12N 15/85; C12Q 1/25; C12Q 1/6809; C12Q 1/6883; G01N 33/5023; G01N 33/5061; C12N 2310/14; C12N 2310/531; C12N 2503/02; C12N 2510/00; C12Y 603/05004; G01N 2333/9015; G01N 2500/10

(57) ABSTRACT

The present invention relates to the Asparagine Synthetase (Asns) gene and the role it plays in cardiomyocyte dedifferentiation and cardiac regeneration. More particularly, the invention is directed to the detection of ASNS expression as a diagnostic marker for cardiomyocyte dedifferentiation and cardiac regeneration; therapeutic up- and downregulation of ASNS expression to augment dedifferentiation-regeneration or suppress dedifferentiation-regeneration; and methods of screening for ASNS inhibitors, activators and regulators of cardiomyocyte dedifferentiation activity. Also disclosed are vectors, probes, therapeutics and kits.

### CROSS-REFERENCE TO RELATED APPLICATION

The present application is a filing under 35 U.S.C. 371 as the National Stage of International Application No. PCT/SG2024/050156, filed Mar. 15, 2024, entitled “A NOVEL MARKER FOR CARDIOMYOCYTES DEDIFFERENTIATION,” which claims priority to Singapore Application No. 10202300882X filed with the Intellectual Property Office of Singapore on Mar. 30, 2023, both of which are incorporated herein by reference in their entirety for all purposes.

### INCORPORATION BY REFERENCE OF MATERIAL IN XML FILE

This application incorporates by reference the Sequence Listing contained in the following XML file being submitted concurrently herewith:
File name: 4373-20900_SP106177USZ_Sequence_Listing.xml, created on Sep. 26, 2025; and having a file size of 11.3 KB. The information in the Sequence Listing is incorporated herein in its entirety for all purposes.

### FIELD OF THE INVENTION

The present invention relates to the Asparagine Synthetase (Asns) gene and the role it plays in cardiomyocyte dedifferentiation and cardiac regeneration. More particularly, the invention is directed to the detection of ASNS expression as a diagnostic marker for cardiomyocyte dedifferentiation and cardiac regeneration; therapeutic up- and downregulation of ASNS expression to augment dedifferentiation-regeneration or suppress dedifferentiation-regeneration; and methods of screening for ASNS inhibitors, activators and regulators of cardiomyocyte dedifferentiation activity. Also disclosed are vectors, probes, therapeutics and kits.

### BACKGROUND OF THE INVENTION

The adult mammalian heart does not self-regenerate, due to a limited proliferative capacity of cardiomyocytes (CMs). As a consequence, cardiac injuries cause irreversible loss of cardiac function, and deteriorate to end-stage clinical heart failure. During the past two decades, much attention has focused on promoting endogenous heart regeneration by manipulating key regulators of CM cell cycle, including Hippo-Yap signaling, Nrg1-ErbB signaling, Meis1, and other cell cycle factors. A more extreme, but effective example was by inducing the overexpression of the “Yamanaka factors” (Oct4, Sox2, Klf4, Myc, “OSKM”), targeted to adult mouse CMs in vivo. In all these studies, CM dedifferentiation (CMDD) is observed prior to CM proliferation, as a precursor to heart regeneration, indicated by hallmarks of cellular immaturity, but progressing beyond the typical myocardial stress-response seen otherwise following pressure-overload or in non-regenerative models of heart failure. Regenerative models of CMDD are instead characterized by morphological, cytoskeletal and metabolic changes, such as the loss of sarcomeres and contractility, reduced CM-CM junctions, initiation of cell cycle re-entry, and metabolic switching from fatty acid oxidation to glycolysis. Adult mammalian CMs also remodel and dedifferentiate when cultured in vitro, displaying a transcriptional reversion of CM maturation. Very little is known of the molecular mechanisms underlying the progressive stages of CMDD. Previous studies reported Oncostatin M (OSM) as a major regulator of this process. Dab2 and Runx1 were significantly induced by OSM and proposed as CMDD markers. However, they were confirmed as upregulated in some, but not all subsequent studies involving CMDD and heart regeneration. Whether these two genes are conserved as CMDD markers in other species is also unknown.

Thus, there is still a need to develop a robust lineage tracer for the tracking and documentation of CMDD, and to serve as a potential target for the diagnosis and treatment of heart failure.

### SUMMARY OF THE INVENTION

According to the present invention, Asparagine Synthetase (Asns) was identified and characterized for the first time as a valid robust tracking marker for CMDD both in vitro and in vivo, as it exhibited consistent and conserved upregulation in multiple mouse, zebrafish and pig models of CMDD and heart regeneration, outperforming the known CMDD markers Dab2 and Runx1. This was further validated with a vector comprising an Asns promoter operably linked to a reporter gene Cre recombinase. Further, knocking down Asns using shRNA resulted in increased CM death in vitro and induced less CM cell cycles in vivo, while overexpressing Asns augmented CMDD induced by OSKM, demonstrating the potential use of this marker in the augmentation or suppression of CMDD, as well as in the diagnosis and treatment of CMDD-related diseases, such as heart failure.

In a first aspect, there is provided a method of detecting cardiomyocyte dedifferentiation (CMDD) in a test sample obtained from a subject, comprising:
(a) determining the expression level of an Asparagine Synthetase (Asns) gene, protein or mRNA in the test sample;
(b) comparing the expression level of the Asns gene, protein or mRNA in the test sample to that in a reference sample;
wherein a difference in the level of the Asns gene, protein or mRNA in the test sample compared to the reference sample indicates that the test sample contains dedifferentiating cardiomyocytes.

In a second aspect, there is provided a vector comprising:
i) a polynucleotide sequence comprising an Asns gene promoter operably linked to a reporter gene; or
ii) a promoter operably linked to a polynucleotide sequence encoding an Asns protein.

In a third aspect, there is provided a transgenic mouse comprising an expression cassette comprising an Asns gene promoter operably linked to a reporter gene. Preferably, the transgenic mouse comprises the vector of the second aspect of the invention and/or the reporter gene is heterologous.

In a fourth aspect, there is provided a kit for determining whether a test sample comprises dedifferentiating cardiomyocytes, comprising the vector of the second aspect of the invention, or a binding agent for Asns gene, protein or mRNA.

In a fifth aspect, there is provided a method for augmenting cardiomyocyte dedifferentiation (CMDD), comprising contacting a cardiomyocyte with an expression vector comprising a polynucleotide sequence which increases the expression or activity of Asns.

In a sixth aspect, there is provided a vector comprising a polynucleotide sequence encoding Asns protein.

In a seventh aspect, there is provided a method of suppressing cardiomyocyte dedifferentiation (CMDD) comprising: contacting a cardiomyocyte with an expression vector comprising a polynucleotide sequence which suppresses the expression or activity of Asns.

In an eighth aspect, there is provided a method of screening for a therapeutic or prophylactic drug having cardiomyocyte dedifferentiation-promoting activity, comprising:
(a) contacting a candidate therapeutic or prophylactic drug with cardiomyocytes or HEK293T cells;
(b) detecting the expression level of Asns protein, mRNA, or Asns gene in the presence of the candidate drug; and
(c) Identifying the candidate therapeutic or prophylactic drug as having cardiomyocyte dedifferentiation-promoting activity if the expression level of the Asns protein, mRNA or Asns gene increases compared to that of control cardiomyocytes or HEK293T cells unexposed to the candidate therapeutic or prophylactic drug.

In a ninth aspect, there is provided a method for treating heart failure in a subject in need thereof, comprising administering to the subject a combination of an efficacious amount of:
a) an expression vector comprising a polynucleotide sequence encoding Asns, and
b) (i) an inhibitor of Hippo-Yap signalling or
(ii) an activator of the ErbB2 pathway, or
(iii) an upregulator of the expression of OSKM factors Oct4, Sox2, Klf4 and Myc,
wherein the combination promotes regeneration of heart tissue greater than that induced in the absence of the expression vector.

In a tenth aspect, there is provided a use of a composition comprising an expression vector comprising a polynucleotide sequence encoding Asns in the manufacture of a medicament for the treatment of heart failure.

### BRIEF DESCRIPTION OF THE DRAWINGS

Certain embodiments of the present disclosure are described more fully hereinafter with reference to the accompanying drawings.

FIG. 1 shows representative images of isolated adult mouse cardiomyocytes (ACMs) in vitro cultured from D0 to D14 (n=4 independent CM isolations). Scale bar=200 μm.

FIG. 2 depicts immunofluorescence (IF) images showing the breakdown and re-construction of ACM sarcomeres from D0 to D14, staining for α-Actinin, cTnl and MyBPC3. Scale bar=10 μm.

FIG. 3 shows GO terms enriched in 6 gene modules from bulk RNA sequencing results of cultured ACM.

FIG. 4 shows top transcription factor DNA binding motifs of ETS family enriched in the turquoise gene module (Module 6 in FIG. 3), obtained by HOMER motif analysis.

FIG. 5 shows mRNA expression of Tnnt2, Myh6, Nkx2.5, Gata4, Acta2 normalised to Rplp0, as well Myh7/Myh6 ratio from D0 to D14 by RT-qPCR. n=4 independent isolations at each time point.

FIG. 6A Constructs of AAV9 comprising cTnT-driven Oct4, Sox2, Klf4 and Myc, fused to corresponding His6, Flag, HA and V5 signal peptides. FIG. 6B Three dose gradients (1×, 2×, 4×) and FIG. 6C experimental timeline of AAV9 injections. AAV-cTnT-BFP was injected as control.

FIG. 7A Quantification of OSKM mRNA expression abundance in purified CMs by RT-qPCR. FIG. 7B Representative Western blot and FIG. 7C quantification for OSKM expression in purified CMs.

FIG. 8A Cardiac functional and structural changes during the experimental timeline by echocardiography. n=10 to 14 mice per group per time point. EF %-ejection fraction; IVS; d-interventricular septum thickness at end-diastole; LVID; d-left ventricular internal diameter at end-diastole. FIG. 8B H&E Staining (top row) and Picrosirius red staining (bottom row) of heart sections of mice receiving AAV392 Control and AAV-OSKM (2×). Scale bar=1 mm.

FIG. 9 depicts IF images showing α-SMA on heart sections. α-SMA was restricted to vessel smooth muscle cells (VSMCs) in Control group (left), but present in both VSMCs and CMs in OSKM hearts (right). Scale bar=100 μm and 20 μm, at low and high magnification images, respectively.

FIG. 10A IF images showing the loss of Connexin 43 at CM-CM junctions in OSKM hearts. Scale bar=50 μm and 10 μm, in low and high magnification images, respectively. FIG. 10B mRNA expression of Gja1 in purified CMs by RT-qPCR. FIG. 10C mRNA expression of Myl2, Myh6 and Myh7 in purified CMs by RT-qPCR.

FIG. 11 shows quantification of positively stained CMs. Each data point represents an independent mouse heart.

FIG. 12 shows absolute CM counts measured by dissociating the whole hearts of Saline and OSKM groups. Data were presented as mean±SD. Student's t test was used. n.s. (non-significant), *p<0.05, **p<0.01, compared to Saline.

FIG. 13 shows GO terms (solid bars) and KEGG pathways (hollow bars) enriched in upregulated (right) and downregulated (left) genes in OSKM-CMs.

FIG. 14 shows overlapping DE genes between the cultured-ACMs and OSKM/Control-CMs datasets, and their respective enriched GO terms. From top to bottom: DE genes in OSKM/Control-CMs dataset overlap with the modules from the cultured-ACMs dataset.

FIG. 15 shows a schematic diagram depicting the criteria and cut-off by which Asns was screened out as a marker gene.

FIG. 16A Asns mRNA expression in cultured ACMs by RT-qPCR. n=4 biological replicates. FIG. 16B Quantification of ASNS⁺ CMs in IF staining from D0 to D14. n=3 biological replicates, and more than 200 CMs analyzed for each time point per replicate.

FIG. 17A Representative Western blot and FIG. 17B quantification for ASNS protein expression in purified CMs from Control and OSKM hearts. FIG. 17C Asns mRNA expression in purified Control-CMs and OSKM-CMs by RT-qPCR.

FIG. 18A Representative images of ASNS⁺ CMs on OSKM-heart sections. Scale bar=10 μm. FIG. 18B Quantification of ASNS⁺ CMs per heart section in IF staining. 2-3 heart sections were analyzed per mouse. Each data point represents an independent mouse heart.

FIG. 19A Percentage of cultured ACMs in each category from D0 to D14. More than 100 CMs were analyzed for each time point per isolation. n=3 independent isolations. FIG. 19B Percentage of cultured ACMs in each category, by pooling ACMs from all time points. n=3 independent isolations.

FIGS. 20A-20D Representative IF images containing ASNS+ (solid line arrows) and ASNS⁻ CMs (dash line arrows) classified into FIG. 20A Cat #1, FIG. 20B Cat #2 and FIG. 20C Cat #3. Scale bar=10 μm. FIG. 20D Percentage of ASNS⁺ and ASNS⁻ CMs displaying organised and disorganised sarcomeres by pooling OSKM and Control groups, from Cat #2 and #3 in FIGS. 20A-20D.

FIG. 21A Percentage of Ki67⁺ and Ki67⁻ CMs in the subpopulations of ASNS⁺ and ASNS-CMs. More than 200 CMs from 5 OSKM-heart sections were analyzed. FIG. 21B Average nuclear number (indicated by DAPI staining) in ASNS⁺ and ASNS⁻ CMs (n=38 CMs) by pooling both OSKM and Control groups.

FIG. 22A A schematic diagram showing Asns promoter located at mouse genome, which was cloned upstream of the Firefly luciferase (luc) reporter. luc driven by SV40 promoter and luc without any upstream promoter (Null) were positive and negative controls for luc expression, respectively. FIG. 22B Relative Firefly luminescence (normalized to Renilla) showing luc expression in HEK293T cells. FIG. 22C The constructs of AAV9 plasmids comprising Asns- and cTnT-promoter driven Cre (top) and a schematic diagram depicting Cre expression in mTmG^{+/-} mouse (bottom). FIG. 22D The experimental timeline of AAV9 or saline injection into adult mTmG+/− mice, followed by CM isolation.

FIGS. 23A-23B show percentages of GFP⁺ FIG. 23A and RFP⁺ FIG. 23B CMs from D0 to D14. n=3 independent isolations, with more than 100 CMs analyzed at each time point per group per isolation.

FIG. 24A mRNA expression of Sox2, Klf4, Myc in mTmG^{+/-} mouse heart tissues by RT-qPCR. FIG. 24B Quantification of GFP⁺ CMs on mTmG^{+/-} mouse heart sections. More than 400 CMs were analyzed per heart. mRNA expression of iCre FIG. 24C and GFP FIG. 24D in mTmG^{+/-} mouse heart tissues by RT-qPCR. Student's t test was used between two groups: n.s. (non-significant), *p<0.05.

FIG. 25A Experimental timeline of Asns^{KD} in adult wild type mouse and FIG. 25B echocardiographic results for up to 8 weeks post AAV9 injection.

FIG. 26A Asns mRNA expression in cultured ACMs by RT-qPCR. FIG. 26B Quantification of PI⁺ cultured ACMs on D4. More than 600 CMs were analyzed per isolation per group.

FIG. 26C The rate of CM loss from D0 to D4 by counting the number of cultured ACMs.

FIG. 27 depicts heatmaps showing key metabolic pathways (fructose metabolism, glycolysis, TCA cycle, FA metabolism) quantified by metabolomics analysis in cultured ACMs on D0 and D4. WT-LacZ-shRNA, KD-Asns-shRNA. n=5 to 6 per group. Quantification was normalized to ACM counting.

FIG. 28A Heatmaps showing (a) the mean and (b) individual values of amino acids quantified by metabolomic profiling of cultured ACMs on D0 and D4. WT-LacZ-shRNA, KD-Asns shRNA. n=5 to 6 per group. Amino acid quantification was normalised to ACM count. FIG. 28B Quantification of PI⁺ CMs on D4, after Asn, Gln and asparaginase (ASPG) treatment.

FIG. 29 shows mRNA expression of Asns and other cell cycle genes (Cdk1, Mki67, Aukrb) in cultured ACMs by RT-qPCR.

FIG. 30A mRNA expression of OSKM in 4F+Asns^{KD} heart tissues, by RT-qPCR. FIG. 30B RT-qPCR validating the efficiency of Asns KD at mRNA expression level in heart tissues.

FIG. 31 shows percentage of CMs stained positive for Ki67, pH3 and Aurora kinase B, respectively. Three heart sections were analyzed per mouse heart. Each data point represents an independent mouse heart.

FIGS. 32A-32D show quantification of amino acids, Asn, Gln, Glu and Asp (normalised to total protein) in heart tissues, measured by LC-MS. Data were presented as mean±SD. One-way ANOVA followed by multiple comparisons was used. Multiple comparisons: n.s. (non-significant), *p<0.05, **p<0.01, ***p<0.001, ****p<0.0001.

FIG. 33 shows cardiac function and structure at Week 4 after AAV9-AsnsOE injection, measured by echocardiography.

FIG. 34 shows mRNA expression of Asns, OSKM, Myh6, Myh7 and Acta2 in OSKM^{1×}Asns^{OE} heart tissues, by RT-qPCR.

FIG. 35 shows cardiac functional and structural changes measured by echocardiography.

FIG. 36 shows percentage of CMs stained positive for Ki67, pH3 and Aurora kinase B, respectively. Three heart sections were analyzed per mouse heart. Each data point represents an independent mouse heart.

FIG. 37 shows quantification of amino acids, Asn, Gln, Glu and Asp (normalised to total protein) in heart tissues, measured by LC-MS.

FIG. 38A Western blot and FIG. 38B quantification showing GLUL, p-S6K1 and p-4EBP1 in 4F+Asns^{KD} mouse heart tissues. Numbers 1 to 4 represent 4 individual mice.

FIG. 39A Representative Western blot and FIG. 39B quantification showing GLUL, p-S6K1 and p-4EBP1 in cultured ACMs with Asns KD. FIG. 39C Protein-protein interaction network of mouse Asns, Glul and mTORC1 pathway related genes (Mtor, Rheb, Rps6 kb1, Eif4ebp1), from the STRING database.

FIG. 40A Experimental timeline of MI in neonatal mouse and sample collection. FIG. 40B IF staining of ASNS in the border zone of MI heart on D7 post MI. Scale bar=1000 μm and 50 μm in low and high magnification images, respectively. FIG. 40C The experimental timeline of MI and AAV9-shRNA injection in neonatal mouse, to suppress Asns, and sample collection. FIG. 40D Immunofluorescent staining results showing GFP reporter on D7 post-surgery. Scale bar=1000 μm and 50 μm in low and high magnification images, respectively.

FIG. 41A Quantification of ASNS expression at the border zone of MI heart on D7. FIG. 41B Quantification of cardiomyocytes stained positive for pH3. FIG. 41C Quantification of cardiomyocytes stained positive for Aurora kinase B.

FIGS. 42A-42B Picrosirius red staining of heart sections collected on D21 post-surgery. Scale bar=1000 μm. FIG. 42C Quantification of fibrotic scars in heart sections collected on D21 post-surgery, showing that regeneration failed in Asns suppressed hearts.

FIG. 43 shows a diagram of high-throughput screening for small molecular and genetic Asns inhibitors, activators and regulators.

### DETAILED DESCRIPTION OF THE INVENTION

Further details of the invention will now be described with reference to the following non-limiting examples. Unless otherwise defined herein, technical and scientific terms used in the present description have the meanings that are commonly understood by those of ordinary skill in the art.

### A. Definitions

As used herein, the singular forms “a,” “an,” and “the” include plural referents unless the context clearly dictates otherwise.

As used herein, the term “comprising” may include the embodiments “consisting of” and “consisting essentially of”. The terms “comprise(s),” “include(s),” “having,” “has,” “can,” “contain(s),” and variants thereof, as used herein, are intended to be open-ended transitional phrases that require the presence of the named features/steps and permit the presence of other features/steps. However, such description should be construed as also describing compositions, mixtures, or processes as “consisting of” and “consisting essentially of” the enumerated features/steps, which allows the presence of only the named features/steps, along with any impurities that might result therefrom, and excludes other features/steps.

As used herein, the term “therapeutically effective amount” or “efficacious amount” of a drug refers to an amount of the drug that is an amount sufficient to obtain a pharmacological response such as activating a biological target (e.g., increasing the expression or activity of Asns); or alternatively, is an amount of the drug that, when administered to a subject with a specified disorder or disease, is sufficient to have the intended effect, e.g., treatment, alleviation, amelioration, palliation or elimination of one or more manifestations of the specified disorder or disease in the subject. A therapeutic effect does not necessarily occur by administration of one dose, and may occur only after administration of a series of doses. Thus, a therapeutically effective amount may be administered in one or more administrations. The therapeutically effective amount will depend in part on the nature of the drug, the manner and route of administration, the stage and severity of the disease being treated, the weight and general state of health of the subject, and the judgment of the prescribing physician. Further, when the term “therapeutically effective amount” or “efficacious amount” is used to refer to a synergistic combination of antibiotic compounds, it means that each of said compounds is provided at an amount such that the combination, as a whole, would provide therapeutic effects. It would be understood that, for said synergistic combination of antibiotic compounds, the amount of one or both of said compounds in the combination, if used alone at the dose in the combination, may not provide a therapeutic effect.

As used herein, the term “subject” refers to animals, typically mammals. Any suitable mammal can be treated by a method described herein. Non-limiting examples of mammals include humans, non-human primates, domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has or is diagnosed of having a particular disease, for example, heart failure.

Where a range of values is provided, it is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

The term “biomarker” is used herein to refer to a molecule whose level of nucleic acid or protein product has a quantitatively differential concentration or level with respect to an aspect of a biological state of a subject. “Biomarker” is used interchangeably with “marker” herein. The level of the biomarker can be measured at both the nucleic acid level as well as the polypeptide level. At the nucleic acid level, a nucleic acid gene or a transcript which is transcribed from any part of the subject's chromosomal and extrachromosomal genome, including for example the mitochondrial genome, may be measured. Preferably an RNA transcript, more preferably an RNA transcript includes a primary transcript, a spliced transcript, an alternatively spliced transcript, or an mRNA of the biomarker is measured. At the polypeptide level, a pre-propeptide, a propeptide, a mature peptide or a secreted peptide of the biomarker may be measured. A biomarker can be used either solely or in conjunction with one or more other identified biomarkers so as to allow correlation to the biological state of interest as defined herein. Specifically, biomarkers of the present invention include Asparagine Synthetase (Asns).

The term “expression level” as used herein refers to detecting the amount or level of expression of a biomarker (i.e., Asns gene) of the present invention. The act of actually detecting the expression level of a biomarker can include determining whether the biomarker expression is present or absent in a sample, and whether it is upregulated, downregulated or substantially unchanged as compared to a control level expressed in a sample or a level expressed in a control sample. The expression level in some cases may refer to detecting transcription of the gene encoding a biomarker protein and/or to detecting translation of the biomarker protein.

A “reference sample” or “control sample,” as used herein, refers to a sample that is used as basis for comparison purposes. In one embodiment, a reference sample, or control sample is obtained from a healthy and/or non-diseased part of the body (e.g., tissue or cells) of the same subject or individual. For example, the reference sample or control sample may be healthy and/or non-diseased cells or tissue adjacent to the diseased cells or tissue (e.g., cells or tissue with heart failure). In another embodiment, a reference sample is obtained from an untreated tissue and/or cell of the body of the same subject or individual. In yet another embodiment, a reference sample or control sample is obtained from a healthy and/or non-diseased part of the body (e.g., tissues or cells) of an individual who is not the subject or individual. In even another embodiment, a reference sample or control sample is obtained from an untreated tissue and/or cell of the body of an individual who is not the subject or individual. In another embodiment, a reference sample or control sample refers to a sample that has been confirmed as not comprising certain specific components. For example, a reference sample or control sample may be a sample comprising no dedifferentiating cardiomyocytes, such as those derived from a healthy heart (i.e., has no heart failure).

The term “promoter” as used herein refers to refers to a DNA sequence capable of controlling the expression of a coding sequence or functional RNA.

The term “operably linked” as used herein refers to a first molecule that can be joined to a second molecule, wherein the molecules are so arranged that the first molecule affects the function of the second molecule. The term “operably linked” includes the juxtaposition of two or more components (e.g., a promoter and another sequence) such that both components function normally and allow for the possibility that at least one of the components can mediate a function that is exerted upon at least one of the other components. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. In additional embodiments, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

The term “endogenous” as used herein describes a molecule (e.g., a polypeptide, nucleic acid, or cofactor) that is found naturally in a particular organism (e.g., a human) or in a particular location within an organism (e.g., an organ, a tissue, or a cell, such as a human cell, e.g., a human hair cell).

The term “exogenous” as used herein describes a molecule (e.g., a polypeptide, nucleic acid, or cofactor) that is not found naturally in a particular organism (e.g., a human) or in a particular location within an organism (e.g., an organ, a tissue, or a cell, such as a human cell, e.g., a human hair cell). Exogenous materials include those that are provided from an external source to an organism or to cultured matter extracted there from.

The term “vector” or “expression vector” as used herein includes a nucleic acid vector, e.g., a DNA vector such as a plasmid, cosmid, or artificial chromosome, an RNA vector, a virus, or any other suitable replicon (e.g., viral vector). A variety of vectors have been developed for the delivery of polynucleotides (DNA or RNA sequences) into a prokaryotic or eukaryotic cell. Specifically, vector suitable for use in the present invention is a virus vector, such as adenoviruses, retroviruses, lentiviruses, adeno-associated viruses (AAV), preferably is AAV. More preferably, vectors are AAVs serotypes capable of targeting cardiomyocytes, such as AAV1, AAV4, AAV6, AAV7, AAV8, AAV9 or AAV-rh10. In a specific embodiment, the vector is AAV9.

The term “reporter gene” refers to a gene that is attached to a regulatory sequence (e.g. a promoter) of another gene of interest and confers organisms expressing it easily identified and measured characteristics. Reporter genes can be used to track the physical location of a segment of DNA or to monitor gene expression. There are generally two types of reporter genes, nonfluorescent type (e.g., an enzyme) and fluorescent type. Chloramphenicol acetyltransferase (CAT), Cre recombinase (Cre), luciferase and lacZ gene are commonly employed as nonfluorescent reporter genes. Examples of fluorescent reporter genes are green fluorescent protein (GFP) and red fluorescent protein (RFP) as well as their variants, such as yellow fluorescent protein (YFP), cyan fluorescent protein (CFP), enhanced yellow fluorescent protein (EYFP), enhanced cyan fluorescent protein (ECFP), enhanced green fluorescent protein (EGFP), monomeric cherry (mCherry) and tandem dimer Tomato (tdTomato), and Lucigen Yellow (LucY). In some embodiments, the combination of nonfluorescent reporter genes with fluorescent protein-coding genes into a single sequence are used and provid an additional tool for analyzing cell populations in vivo and ex vivo, because this strategy allows 2 signals to be monitored independently.

### B. Use of Asns for Detecting CMDD

In a first aspect, the present invention provides a method of detecting cardiomyocyte dedifferentiation (CMDD) in a test sample obtained from a subject, comprising:
(a) determining an expression level of an Asparagine Synthetase (Asns) gene, protein or mRNA in the test sample;
(b) comparing the expression level of the Asns gene, protein or mRNA in the test sample to that in a reference sample;
wherein a difference in the level of the Asns gene, protein or mRNA in the test sample compared to the reference sample indicates that the test sample contains dedifferentiating cardiomyocytes.

In some embodiments, a significantly increased level of the Asns gene, protein or mRNA in the test sample compared to the reference sample indicates that the test sample contains dedifferentiating cardiomyocytes. Any statistical analysis method known in the art may be used in the present invention to determine whether the difference between the test sample and the reference sample achieves a statistically significant level, such as Student's t test.

In some embodiments, the reference sample suitable for use in the CMDD detection method of the present invention refers to a cardiac sample comprising no dedifferentiating cardiomyocytes. In some embodiments, the reference sample is obtained from a heart that is not undergoing cardiac regeneration or that comprises no cardiomyocyte dedifferentiation, such as a normal heart.

In some embodiments, the test sample comprises isolated cardiomyocytes from a cardiac tissue or from a biopsy sample. In some embodiments, the reference sample is obtained from a healthy heart that does not show CMDD.

In some embodiments, the CMDD is in the progression to achieve cardiac regeneration. As CMDD is believed to be a signal of heart regeneration, the detection of CMDD in a test sample means that the test sample is undergoing heart regeneration.

Step (a) determining the expression level of Asns gene, protein, or mRNA can be performed according to any method well known in the art. In general, expression of a nucleic acid molecule (e.g. mRNA or DNA) can be detected by any suitable method or technique of measuring or detecting a gene or polynucleotide sequence or the expression thereof. Such methods include, but are not limited to, polymerase chain reaction (PCR), reverse transcriptase PCR (RT-PCR), in situ PCR, quantitative PCR (q-PCR), quantitative RT-PCR (qRT-PCR), in situ hybridization, Southern blot, Northern blot, sequence analysis, microarray analysis, DNA/RNA-sequencing, detection of a reporter gene, or any other DNA/RNA hybridization platforms. Generally, these methods involve the use of gene-specific (such as Asns) oligonucleotide primers or probes, or vectors comprising an Asns gene promoter operably linked to a detectable heterologous reporter gene.

In some embodiments, determining the expression level of Asns gene, protein or mRNA comprises using:
i) an antibody which specifically binds to Asns protein;
ii) an Asns-specific oligonucleotide primer;
iii) an Asns-specific oligonucleotide probe; or
iv) an expression vector comprising an Asns gene promoter operably linked to a detectable heterologous reporter gene.

In some embodiments, the Asns gene promoter is a mouse or human Asns gene promoter. Preferably, the Asns gene promoter comprises a nucleic acid sequence having at least 85%, 90%, 91%, 92%, 93%, 94%, 95%, 96%, 97%, 98%, 99%, or 100% sequence identity with SEQ ID NO: 1 or SEQ ID NO: 2. More preferably, the mouse Asns gene promoter comprises the nucleic acid sequence set forth in SEQ ID NO: 1 and the human Asns gene promoter comprises the nucleic acid sequence set forth in SEQ ID NO: 2.

Reporter genes well known in the art and suitable for use in gene expression may be used in the methods of the present invention. In some embodiments, the reporter gene may encode an enzyme or a fluorescent protein. Preferably, the reporter gene encodes at least one protein selected from the group comprising CAT, Cre recombinase, luciferase, lacZ, GFP, RFP, YFP, CFP, EYFP, ECFP, EGFP, mCherry, tdTomato, LucY and combinations thereof. More preferably, the reporter gene encodes a Cre recombinase.

In some embodiments, the vector is a DNA vector such as plasmid, cosmid, or artificial chromosome, an RNA vector, or a virus, preferably a virus vector. In a preferable embodiment, the vector is a virus vector selected from a group comprising adenoviruses, retroviruses, lentiviruses, and adeno-associated viruses (AAV). More preferably, the vector is an AAV vector, such as AAV1, AAV4, AAV6, AAV7, AAV8, AAV9 or AAV-rh10. In a specific embodiment, the vector is AAV9.

In embodiments wherein the vector is a virus vector, the dosages to be administered in methods of the invention will vary depending, for example, on the particular virus type, the mode of administration, the treatment goal, the individual, and the cell type(s) being targeted. It may be determined as needed by a skilled person in the art using well known methods. The dosages may be expressed in units of DNase resistant particles (DRP) per ml or viral genomes (vg). For example, in some in vitro culture systems, the dosages for virus such as AAV and adenovirus may be in the range from 1000 to 3000 vg/cell, preferably from 1000 to 2500 vg/cell, from 1500 to 2500 vg/cell, from 1800 to 2200 vg/cell, and more preferably around 2000 vg/cell. Unlike AAV, the lentiviral vector may integrate into the host genome, thus the dosage needs to keep low to ensure each cell is infected by up to only one virus and therefore, only one copy of the lentiviral vector will be integrated into the host genome. Thus, the dosages for virus such as lentivirus may be in the range from 5000 to 10000 vg/10000 cells. For some in vivo administration in mouse, the dosage may range from about 1.25×10¹³ to 5×10¹³ vg/kg body weight, preferably from 1.5×10¹³ to 4.5×10¹³ vg/kg body weight, from 2×10¹³ to 4×10¹³ vg/kg body weight, from 2.5×10¹³ to 3×10¹³ vg/kg body weight, and more preferably around 2.5×10¹³ vg/kg body weight. For in vivo administration in human, the dosage of AAV in current clinical trials varies widely depending on several factors including the target disease, the AAV serotype (i.e., the specific subtype of AAV being used), the route of administration (e.g., intravenous, intramuscular, subretinal). The dosing can range from low doses in the order of 109 viral genomes (vg) per kilogram of body weight (e.g. regional delivery such as retina-related diseases) to high doses exceeding 10¹⁴ vg/kg in some instances. Therefore, the dosage for human may range from 1×10¹³ to 1×10¹⁴ vg/kg of body weight.

In some embodiments, the viral vectors are packed into virus to be administered in the following ranges:
a) 1.25×10¹³ vg/kg of body weight to 5×10¹³ vg/kg of body weight, for mouse, and 1×10¹³ to 1×10¹⁴ vg/kg of body weight for human in vivo, or
b) 1000-3000 vg/cell, for AAV and adenovirus in cultured cells in vitro, or
c) 5000 to 10000 vg/10000 cells, for lentivirus in cultured cells in vitro.

In some embodiments, the expression level of Asns protein may be determined by methods including, but not limited to: Western blot, immunoblot, enzyme-linked immunosorbent assay (ELISA), radioimmunoassay (RIA), immunoprecipitation, surface plasmon resonance, chemiluminescence, fluorescent polarization, phosphorescence, immunohistochemical analysis, liquid chromatography mass spectrometry (LC-MS), matrix-assisted laser desorption/ionization time-of-flight (MALDI-TOF), mass spectrometry, microcytometry, microarray, microscopy, fluorescence activated cell sorting (FACS), flow cytometry, and assays based on a property of the protein including but not limited to DNA binding, ligand binding, or interaction with other protein partners. The detection of Asns protein expression level may involve the use of antibodies that specifically recognize and bind to Asns.

Small molecules that specifically bind to Asns protein or mRNA may also be used to determine the expression of Asns. For example, such small molecules attached with an enzyme or a fluorescent protein or an isotope label may be cultured with the test sample for a period and then the intensity/quantity of the enzyme/fluorescent protein/isotope is determined, thereby indirectly determining the expression level of Asns gene, protein or mRNA.

In some embodiments, the expression level of Asns gene, protein or mRNA in the test sample and in the reference sample may be detected at the same time using the same method and then compared with each other. In other embodiments, the expression level of Asns gene, protein or mRNA in the test sample may be compared with the pre-determined level of the corresponding Asns gene, protein or mRNA in the reference sample. In such embodiment, the pre-determined level of Asns gene, protein or mRNA in the reference sample is preferably determined by the same method used for the test sample.

In a second aspect, the present invention also provides a vector comprising an Asns gene promoter operably linked to a detectable heterologous reporter gene, wherein the terms “vector”, “promoter” and “reporter gene” are as defined above in the first aspect of the invention.

In a third aspect, the present invention further provides a transgenic mouse comprising an expression cassette comprising an Asns gene promoter operably linked to a heterologous reporter gene, wherein the terms “promoter” and “reporter gene” are as defined above in the first aspect of the invention.

In some embodiments, the transgenic mouse comprises a vector as defined in the second aspect of the invention.

In a fourth aspect, the present invention further provides a kit for determining whether a test sample comprises dedifferentiating cardiomyocytes, comprising the vector of the second aspect of the invention, or at least one binding agent for Asns promoter, protein or mRNA.

As used herein, the term “binding agent” refers to any entity that is able to specifically bind to a target (i.e., Asns promoter, protein or mRNA). Such binding agent may be an antibody or a functional fragment thereof, an aptamer, or a small molecule. The binding agent may be operably linked to a fluorescent, an enzyme, or an isotope label, which enables the detection of the binding agent after its binding to the target.

### C. Use of Asns for Regulating CMDD

As the expression level of Asns closely relates to the status of CMDD, it is possible to control the status of CMDD, thereby controlling the progression of cardiac regeneration, by regulating (augmenting or suppressing) the expression level of Asns.

Thus, in a fifth aspect, the present invention provides a method for augmenting cardiomyocyte dedifferentiation (CMDD), comprising contacting a cardiomyocyte with an expression vector comprising a polynucleotide sequence which increases the expression or activity of Asns for a period of time.

In some embodiments, the expression vector is a DNA vector such as plasmid, cosmid, or artificial chromosome, an RNA vector, or a virus, preferably a virus vector. In a preferred embodiment, the vector is a virus vector selected from a group comprising adenoviruses, retroviruses, lentiviruses, and adeno-associated viruses (AAV). More preferably, the vector is an AAV vector, such as AAV1, AAV4, AAV6, AAV7, AAV8, AAV9 or AAV-rh10. In a specific embodiment, the vector is AAV9.

In some embodiments, the expression vector comprises a polynucleotide sequence encoding an Asns protein, wherein the polynucleotide sequence is operably linked to a promoter. Preferably, the Asns protein is a mouse or human Asns protein. More preferably, the Asns protein is a mouse Asns protein comprising an amino acid sequence as set forth in SEQ ID NO: 3, or a human Asns protein comprising an amino acid sequence as set forth in SEQ ID NO: 4.

In some embodiments, the polynucleotide sequence is operably linked to a promoter. In some embodiments the promoter is a cardiomyocyte-specific promoter, such as cardiac troponin T (cTnT) promoter, Myl2 promoter, Myh6 promoter, or Asns gene promoter.

In some embodiments the promoter is an Asns gene promoter comprising a nucleic acid sequence having at least 85%, 90%, 91%, 92%, 93%, 94%, 95%, 96%, 97%, 98%, 99%, or 100% sequence identity with SEQ ID NO: 1 or SEQ ID NO: 2. More preferably, the mouse Asns gene promoter comprises the nucleic acid sequence set forth in SEQ ID NO: 1 and the human Asns gene promoter comprises the nucleic acid sequence set forth in SEQ ID NO: 2.

In some embodiments, augmentation of CMDD may be used to enhance cardiac regeneration.

The above-defined expression vector comprising a polynucleotide sequence encoding Asns protein is also provided as a subject matter of the sixth aspect of the present invention.

In a seventh aspect, the present invention provides a method for suppressing cardiomyocyte dedifferentiation (CMDD), comprising contacting a cardiomyocyte with an expression vector comprising a polynucleotide sequence which suppresses the expression or activity of Asns for a period of time.

In some embodiments, suppression of CMDD may be used to control cardiac regeneration, for example, to inhibit or slow down the progression of cardiac regeneration.

In some embodiments, the expression vector comprising a polynucleotide sequence which encodes a noncoding RNA (ncRNA) targeting Asns. Alternatively, the expression vector comprises a polynucleotide sequence comprising a ncRNA targeting Asns.

As used herein, the term “noncoding RNA” or “ncRNA” refers to RNA molecules that are generated from the larger part of the genome that does not encode proteins but produces noncoding transcripts that regulate gene expression and protein function. Examples of ncRNA may be used in the present invention include but not limited to microRNA (miRNA), long ncRNA (lncRNA), RNA interference (RNAi), antisense oligonucleotide (ASO), small interfering RNA (siRNA), short hairpin RNAs (shRNA), ASO anti-microRNA (anti-miR), miRNA mimics, miRNA sponge, therapeutic circular RNA (circRNA), single guide RNA (sgRNA), and CRISPR (clustered, regularly interspaced short palindromic repeats) RNA (crRNA).

miRNAs or microRNAs are highly conserved, small, 17- to 25-nucleotide (nt), single-stranded ncRNAs that act as gene regulators. They function by integrating into the RNA-induced silencing complex (RISC) to mediate either translational repression or mRNA degradation depending on the extent of complementarity to the mRNA target. Translational repression is mediated through effects on translation initiation, elongation and termination as well as co-translational degradation. mRNA degradation is mediated through mechanisms resulting first in mRNA deadenylation, followed by de-capping and concluded by exonuclease-mediated 5′ to 3′ degradation.

Long noncoding RNAs or lncRNAs are a diverse class of RNA molecules >200 base pairs of length that have no coding capacity. LncRNAs primarily interact with mRNA, DNA, protein, and miRNA and consequently regulate gene expression at the epigenetic, transcriptional, post-transcriptional, translational, and post-translational levels in a variety of ways. LncRNAs contain two types of functional element, the interactor elements involved in direct physical interaction with other nucleic acids, with proteins or lipids, and the structural elements, leading to the occurrence of secondary and/or tertiary 3D RNA structures, which direct their functional interactions.

Antisense oligonucleotides or ASOs are single-stranded DNA or RNA molecules with full complementarity to a select target sequence and may act by blocking protein translation (via steric hindrance), causing mRNA degradation (via RNase H cleavage) or changing pre-mRNA splicing (via interference with cis-splicing elements causing exon inclusion or exclusion).

Small interfering RNAs or siRNAs, also known as short interfering RNA or silencing RNA, are a class of double-stranded RNAs, typically 20-24 (normally 21) base pairs in length, and operating within the RNA interference (RNAi) pathway.

Short hairpin RNAs or shRNAs are artificial RNA molecules with a tight hairpin turn that can be used to silence target gene expression via RNA interference.

MiRNA mimics exploit the main advantage of endogenous miRNAs being able to target multiple mRNAs at once. miRNA mimics have the same sequence as an endogenous miRNA while the passenger strand carries a few mismatches to prevent RISC loading and potential action as an anti-microRNA (anti-miR).

Anti-miRs are essentially ASOs designed to be fully or partially complementary to an endogenous miRNA to prevent the interaction with its target genes. Anti-miRs may also be referred to as ‘antagomiRs’ if they are conjugated to cholesterol to improve intracellular delivery.

MiRNA sponges are artificial transcripts that contain multiple miRNA binding sites to trap and sequester it. MiRNA sponges may target one specific or multiple different miRNAs, for instance, to simultaneously inhibit mir-21, miR-155 and miR-221/miR-222 in tumour cells, or they may target a whole miRNA seed family, for instance, to sequester miR-181a, miR-181b and miR-181c.

CRISPR/Cas system is another well-known technique commonly used to edit (e.g., knock out) a target gene or cleave a target RNA, thus inhibiting the expression thereof. In such system, sgRNA refers to a combined RNA consisting of a tracrRNA and at least one crRNA. The crRNA or the crRNA part of the sgRNA directs the Cas enzyme and recognizes the target sequence through the complementary base pair component, and then the Cas enzyme cleaves the target sequence at a desired site upstream to a protospacer adjacent motif (PAM). PAM sequence is a short (2-5 base-pair length) conserved DNA sequence downstream to the cut site and its size varies depending on the bacterial species. Many online tools are available to aid in designing effective sgRNA sequences.

Design strategy of the above ncRNAs as well as methods for detecting the targeting efficiency/inhibition activity of the designed ncRNAs against the target sequence (e.g., Asns gene or mRNA) are well known in the art.

In some embodiments, the ncRNA targeting Asns is selected from a group comprising miRNA, lncRNA, ASO, siRNA, shRNA, antimiR, miRNA mimics, miRNA sponge, therapeutic circular RNA (circRNA), sgRNA, and crRNA. Preferably, the ncRNA targeting Asns is miRNA, siRNA, shRNA, sgRNA or crRNA. More preferably, the ncRNA targeting Asns is siRNA or shRNA.

### D. Use of Asns for Drug Screening

In an eighth aspect, the present invention provides a method method of screening for a therapeutic or prophylactic drug having cardiomyocyte dedifferentiation-promoting activity, comprising:
(a) contacting a candidate therapeutic or prophylactic drug with cardiomyocytes or HEK293T cells;
(b) detecting the expression level of Asns protein, mRNA, or Asns gene in the presence of the candidate drug; and
(c) Identifying the candidate therapeutic or prophylactic drug as having cardiomyocyte dedifferentiation-promoting activity if the expression level of the Asns protein, mRNA or Asns gene increases compared to that of control cardiomyocytes or HEK293T cells unexposed to the candidate therapeutic or prophylactic drug.

In some embodiments, the method for screening a valid candidate drug for cardiomyocyte dedifferentiation-promoting activity, comprises the following steps:
(a) culturing cardiomyocytes with a candidate drug for a period of time;
(b) determining the expression level of Asns protein, mRNA, or Asns gene in the cardiomyocytes cultured with the candidate drug; and
(c) identifying the candidate drug as a valid candidate drug if the expression level of the Asns protein, mRNA or Asns gene increases compared to that of control cardiomyocytes unexposed to the candidate drug.

In some embodiments, before the contacting step, the cardiomyocytes may be transformed with a vector comprising a polynucleotide sequence comprising a heterologous Asns gene promoter operably linked to a reporter gene.

In some embodiments, step (b) of detecting may be performed by the method described in the first aspect of the invention.

In some embodiments, the method is performed in vitro.

### E. Use of Asns for Treating a Disease

As Asns is able to augment CMDD, it may be used to treat heart failure in a subject having a heart undergoing CMDD. It may also be used in combination with a therapy known to initiate or activates CMDD, such as a therapy including Agin, miR-590, miR-199a, or miR302-367, which is known to knockdown the Hippo signalling (see Ikeda et al., 2019; Leach et al., 2017; Morikawa et al., 2015), or a therapy including Nrg1, which is known to activate the ErbB2 pathway (See D'Uva et al., 2015), or a therapy able to upregulate the expression of OSKM factors (See Chen et al., 2021).

In a ninth aspect, the present invention provides a method for treating heart failure in a subject in need thereof, comprising administering to the subject a combination of an efficacious amount of:
a) an expression vector comprising a polynucleotide sequence encoding Asns, and
b) (i) an inhibitor of Hippo-Yap signalling or
(ii) an activator of the ErbB2 pathway, or
(iii) an upregulator of the expression of OSKM factors Oct4, Sox2, Klf4 and Myc, wherein the method promotes regeneration of heart tissue greater than that induced in the absence of the Asns expression vector.

In some embodiments, the therapy inhibiting the Hippo-Yap Signalling pathway includes Agrin, miR-590, miR-199a, or miR302-367. In some embodiments, the therapy activating the ErbB2 pathway includes Neuregulin 1 (Nrg1). In some embodiments, the therapy upregulating the expression of the OSKM factors includes overexpression of the OSKM factors.

In some embodiments, the polynucleotide sequence encodes a human or a mouse Asns protein. Preferably, the polynucleotide sequence comprises an amino acid sequence set forth in SEQ ID NO: 3 or SEQ ID NO: 4.

In some embodiments, the polynucleotide sequence encoding Asns is operably linked to an Asns promoter. Preferably, the Asns promoter comprises a nucleic acid sequence having at least 85%, 90%, 91%, 92%, 93%, 94%, 95%, 96%, 97%, 98%, 99%, or 100% sequence identity with SEQ ID NO: 1 or SEQ ID NO: 2.

In some embodiments, the vector is a DNA vector such as a plasmid, cosmid, or artificial chromosome, an RNA vector, or a virus, preferably a virus vector. In a preferred embodiment, the vector is a virus vector selected from a group comprising adenoviruses, retroviruses, lentiviruses, and adeno-associated viruses (AAV). More preferably, the vector is an AAV vector, such as AAV1, AAV4, AAV6, AAV7, AAV8, AAV9 or AAV-rh10. In a specific embodiment, the vector is AAV9.

In a tenth aspect, the present invention provides a use of a composition comprising an expression vector comprising a polynucleotide sequence encoding Asns in the manufacture of a medicament for the treatment of heart failure.

In some embodiments, the medicament is used for combination therapy with a a drug which inhibits Hippo-Yap signalling (such as Agrin, miR-590, miR-199a, miR302-367) and/or activates ErbB2 pathways (for example, by using Neuregulin1 (Nrg1)), or upgregulating expression of the OSKM factors.

It should be understood that any and all embodiments of the present disclosure can be combined with technical features in any other embodiment or multiple other embodiments to obtain additional embodiments under the premise of no conflict. The invention includes such combinations resulting in further embodiments.

### EXAMPLES

The following examples are intended to exemplify the present disclosures and are not limitations of the claimed invention. All molecules, compositions, methods, assays, and results disclosed in the examples and other sections of the specification, figures, and claims form part of the disclosure of the invention.

### Example 1

### Materials and Methods

### Animals

All animal experimental procedures involving C57BL/6J and mTmG strains were approved by the National University of Singapore Institutional Animal Care and Use Committee (IACUC) and undertaken in strict accordance with the Singapore National Advisory Committee for Laboratory Animal Research guidelines. All mice were housed in individually ventilated cages, with sex-matched littermates, under standard conditions. The i4FHeart mice were generated as reported before (see Haenebalcke L et al., 2013), and were injected with AAV9 in accordance with the Guide for the Care and Use of Laboratory Animals published by the US National Institutes of Health and according to the regulations issued by the Committee for Animal Rights 5 Protection of the State of Hessen (Regierungspraesidium Darmstadt).

Adult Mouse CM Isolation and Culture CMs were isolated and purified from 8 to 12-week old adult mice using the protocol published previously (see Ackers-Johnson M et al., 2016). Purified CMs were either directly lysed for RNA and protein extraction, or pre-plated on uncoated cell culture dishes for 1 hour to further remove non-CMs before culture on laminin-coated cell culture plates. During the first two days of culturing (D0 and D1), 1 UM of blebbistatin (Sigma-Aldrich) was added as a specific myosin II inhibitor (see Kabaeva Z et al., 2008). Starting from D2, blebbistatin was removed to accelerate morphological changes of CMs. 10% FBS was included in the culture media during the whole culture procedure, and the small colonies of non-CMs were carefully scraped under a microscope and washed away daily. Images of CMs were captured using the EVOS® FL Cell Imaging System (Thermo Fisher) under different channels.

### HEK293T Cell Culture

HEK293T cells were cultured in DMEM containing 10% FBS and penicillin/streptomycin.

### Counting of Dissociated CM Number

CMs were fixed by 4% paraformaldehyde in Dulbecco's phosphate-buffered saline (DPBS) after being isolated from adult mice. 10 μL of the fixed CM suspension was used for manual cell counting using a hemocytometer under a microscope. The total number of CMs per heart was counted accordingly.

### Heart Harvesting for Weight Measurement, Fixation, Cryosection and Apex for Snap Freezing

Mice were anesthetised by isoflurane inhalation. The heart was arrested in diastole by injection of 500 μL 15% potassium chloride into the inferior vena cava, followed by perfusion with saline solution. After aorta and auricles were trimmed, hearts were dried on clean napkins to remove excess fluid. Heart weight was measured, after which the apex was resected for snap freezing in liquid nitrogen and stored at −80° C. until further processing. The remaining heart was immersed in 3% sucrose with 4% formaldehyde to be fixed overnight at 4° C., then immersed in 30% sucrose with 0.01% sodium azide for post-fixing overnight at 4° C. before embedded in O.C.T. blocks, according to standard procedures. Blocks were sectioned into 5 μm sections by cryostat equipment for further histological analysis.

RNA Extraction from Mouse Apex Tissue

1 mL of TRIzol™ reagent was added to a piece of apex tissue resected from each mouse heart in a 2 mL Sarstedt tube. One single stainless-steel bead (diameter 5 mm) was added to each tube containing apex tissue in TRIzol™. The TissueLyser II (Qiagen) was assembled as per manufacturer's guidelines. The apex tissue was homogenised for 20 s at 30 Hz for two rounds, until no visible tissue debris was observed. Samples were centrifuged briefly to collect tissue debris. The supernatant was collected for total RNA extraction as per manufacturer's protocol.

RNA Extraction from Isolated and Cultured Mouse CMs

For freshly isolated mouse CMs, a total number of about 120,000 CMs (⅛ of total CMs) per heart were lysed by 1 mL TRIzol™ Reagent. To lyse cultured CMs, 1 mL TRIzol™ Reagent was added to each well in 12-well and 6-well cell culture plates. Total RNA extraction was performed as per manufacturer's protocol.

Complementary DNA (cDNA) Synthesis and RT-qPCR

cDNA was synthesised with oligo dT and random primers using the qScript™ cDNA Synthesis Kit. qPCR was performed with PerfeCTa® Low Rox™ ToughMix™ (Quantabio) on a real-time PCR system. All qPCR data were normalised to the expression of housekeeping genes Gapdh and Rplp0.

### Protein Extraction and Quantification

Protein extraction was carried out using 6 M urea lysis buffer with 1 mM dithiothreitol, protease inhibitor and phosphatase inhibitor. For protein extraction from cultured ACMs and purified CMs, lysis buffer was added directly to the cells, followed by vigorous pipetting. For protein extraction from mouse heart apex, a piece of apex tissue resected from each mouse heart was weighted and submerged in 500 μL lysis buffer in a 2 mL Sarstedt tube. One single stainless-steel bead (diameter 5 mm) was added to each tube containing apex tissue in lysis buffer. The TissueLyser II was assembled as per manufacturer's guidelines with pre-chilled adapters. The apex tissue was homogenised for 20 s at 30 Hz for two rounds, until no visible tissue debris was observed. Protein quantification was done using Bio-Rad Protein Assay Kit I as per manufacturer's guidelines.

### Western Blot

The protein lysate with loading buffer was heated at 60° C. for 10 mins before loading onto a 10% SDS-polyacrylamide gel. After transfer, the blotting membrane was blocked in 5% blotting-grade blocker in TBST for 1 hour, followed by primary antibody incubation at 4° C. overnight and secondary antibody incubation at room temperature (R.T.) for 1 hour. The blot was detected by ECL substrate reaction and exposure on a ChemiDoc™ touch imaging system (BioRad). The quantification of protein expression level was performed using Fiji software. All proteins were normalised to GAPDH. Primary antibodies used are listed in Table 1.

### Transfection and Luciferase Reporter Assays

The mouse Asns promoter and cTnT promoter were cloned upstream of the luciferase reporter gene luc2 (Photinus pyralis, Firefly luciferase) in a vector pGL4.10. The vector pGL4.10 itself was used as a negative control, and the vector pGL4.13 (SV40-luc2) was used as a positive control. 5×10⁴ HEK293T cells were co-transfected with 50 ng of each target vector with luc2 and 50 ng of vector pGL4.73 (SV40-hRluc, Renilla luciferase) using jetPRIMER transfection kit, according to manufacturer's instructions. Luciferase activity was measured 48 h after transfection using the Dual-Glo® Luciferase Assay System, according to manufacturer's protocol, and quantified on a GloMax® multi-plate reader. Results were analysed as Firefly luciferase luminescence normalised to Renilla luciferase luminescence.

### AAV9 Viral Production and Purification

An cTnT promoter was cloned into an AAV9 vector to obtain an AAV-cTnT vector. The four factors Oct4, Sox2, Klf4 and Myc with respective tags, as well as blue fluorescent protein (BFP) were individually cloned into the AAV9-cTnT vector (AAV-cTnT-Oct4-His6, AAV-cTnT-Sox2-Flag, AAV-cTnT-KIf4-HA, AAV-cTnT-Myc-V5 and AAV-cTnT-BFP). Asns shRNA (SEQ ID NO: 5) and LacZ shRNA (5′-CUU UAG_CGA CUA AAC ACA UCA G-3′, SEQ ID NO: 6) and green fluorescent protein (GFP) coding sequence were individually cloned into the AAV9-cTnT vector (AAV-cTnT-Asns shRNA-GFP, AAV-cTnT-LacZ shRNA-GFP). The coding sequence of the mouse Asns protein (SEQ ID NO: 7) with V5 tag was cloned into the AAV9-cTnT vector (AAV-cTnT-Asns-V5). The iCre coding sequence was cloned into the AAV-cTnT vector to obtain AAV-Asns-iCre. The mouse Asns promoter (SEQ ID NO: 1) was individually cloned into the AAV-cTnT-iCre to replace the cTnT promoter (AAV-Asns-iCre). Target AAV9 vectors were packaged by a triple transfection method with helper plasmids pAdAF6 (Addgene Catalog #112867) and pAAV2/9n (Addgene Catalog #112865) and purified as previously described (See Wakimoto H et al., 2016).

### AAV9 Injection and Echocardiography

AAV-cTnT-Oct4-His6, AAV-cTnT-Sox2-Flag, AAV-cTnT-KIf4-HA and AAV-cTnT-Myc-V5 (AAV-OSKM) were mixed and administered at a titer of 1.25×10¹³, 2.5×10¹³ and 5.0×10¹³ virus genome (vg)/body kg/factor as three dose gradients (1×, 2×, 4×), via thoracic cavity injection to 8 to 12-week-old male mice, with AAV-cTnT-BFP administered at a comparable titer accordingly as control. AAV-cTnT-iCre and AAV-Asns-iCre were individually administered at a titer of 5×10¹⁰ vg/body gram to 8 to 12-week-old mTmG+/− mice, with or without AAV-OSKM (2×). AAV-cTnT-Asns shRNA-GFP (Asns shRNA) and AAV-cTnT-LacZ shRNA-GFP (LacZ shRNA) were injected into WT and i4FHeart mice at a titer of 5.0×10¹⁰ vg/body gram. AAV-cTnT-Asns-V5 and AAV-cTnT-BFP at a titer of 5×10¹⁰ vg/body gram were individually mixed with AAV-OSKM (1×) for injection into WT mice. Echocardiography was performed to monitor cardiac function following manufacturer's guide for small animal echocardiography. Echocardiography data were analysed with Vevo 2100 Imaging System.

### Propidium Iodide (PI) Staining Assay

PI (final concentration 1 μg/mL) and Hoechst (final concentration 1 μg/mL) was added at a to cultured ACMs, and incubated at 37° C. for 45 mins. The ACMs were immediately imaged by Nikon inverted microscope Eclipse Ti-E. For each well in the cell cultured dishes, 8 to 9 random regions were imaged. PI+ CMs were quantified using Fiji software.

### Histological Analysis and Immunofluorescence Staining

Heart sections were re-hydrated for hematoxylin and eosin (H&E) staining and Picro Sirius red staining, according to the manufacturer's protocol, followed by imaging using TiE fluorescence microscopy system. For immunofluorescence staining, heart sections were incubated in 10 mM citrate buffer (pH 6.0) for antigen retrieval at 55° C. for 15 mins, 70° C. for 15 mins and 100° C. for 20 mins, followed by permeabilisation using 0.5% Triton X-100/DPBS. The sections were then blocked in DPBS containing 5% goat/donkey serum and 0.1% Triton X-100 at R.T. for 1 hour, followed by incubation in primary antibody or WGA at 4° C. overnight and secondary antibody incubation at R.T. for 1 hour. The sections were incubated in 1% Sudan black B in 70% ethanol at R.T. for 7-8 mins to reduce the autofluorescence of heart sections before the final mounting. For immunofluorescence staining on fixed cells, all procedures were the same as those on heart sections, except that the antigen retrieval and incubation in Sudan black solution were not required. Primary antibodies used are listed in Table 1.

### Confocal/Fluorescence Microscopy and Image Analysis

All tissue sections or fixed cells for immunofluorescence staining were imaged using either the Leica SP8 DSL confocal microscope or Nikon inverted microscope Eclipse Ti-E. For each experiment, all samples were processed in the same batch for immunostaining, and microscopy parameters were set to identical settings for all pictures. Images were analysed using the Leica Application Suite X (LAS X) software and Fiji software.

### Global Metabolomics Study of Cultured ACMs and Data Analysis

The global metabolomic study of cultured ACMs was performed and analysed by Metabolon, Inc. Samples were prepared using the automated MicroLab STAR® system from Hamilton Company. Several recovery standards were added prior to the first step in the extraction process for QC purposes. To remove protein, dissociate small molecules bound to protein or trapped in the precipitated protein matrix, and to recover chemically diverse metabolites, proteins were precipitated with methanol under vigorous shaking for 2 min (Glen Mills GenoGrinder 2000) followed by centrifugation. Samples were placed briefly on a TurboVap® (Zymark) to remove the organic solvent. The sample extracts were stored overnight under nitrogen before preparation for analysis. Sample were analysed with Waters ACQUITY ultra-performance liquid chromatography (UPLC) and a Thermo Scientific Q-Exactive high resolution/accurate mass spectrometer interfaced with a heated electrospray ionization (HESI-II) source and Orbitrap mass analyser operated at 35,000 mass resolution. The sample extract was dried then reconstituted in solvents compatible to each of the four methods. Each reconstitution solvent contained a series of standards at fixed concentrations to ensure injection and chromatographic consistency.

The first sample aliquot was analysed using acidic positive ion conditions, chromatographically optimised for more hydrophilic compounds. In this method, the extract was gradient eluted from a C18 column (Waters UPLC BEH C18-2.1×100 mm, 1.7 μm) using water and methanol, containing 0.05% perfluoropentanoic acid (PFPA) and 0.1% formic acid (FA). The second aliquot was also analysed using acidic positive ion conditions, however it was chromatographically optimised for more hydrophobic compounds. In this method, the extract was gradient eluted from the same afore mentioned C18 column using methanol, acetonitrile, water, 0.05% PFPA and 0.01% FA and was operated at an overall higher organic content. The third aliquot was analysed using basic negative ion optimised conditions using a separate dedicated C18 column.

The basic extracts were gradient eluted from the column using methanol and water, however with 6.5 mM Ammonium Bicarbonate at pH 8. The last aliquot was analysed via negative ionization following elution from a HILIC column (Waters UPLC BEH Amide 2.1×150 mm, 1.7 μm) using a gradient consisting of water and acetonitrile with 10 mM Ammonium Formate, pH 10.8.

The MS analysis alternated between MS and data-dependent MSn scans using dynamic exclusion. The scan range varied slighted between methods but covered 70-1000 m/z. Raw data was extracted, peak-identified and QC processed using Metabolon's hardware and software. These systems are built on a web-service platform utilizing Microsoft's .NET technologies, which run on high-performance application servers and fiber-channel storage arrays in clusters to provide active failover and load-balancing. Compounds were identified by comparison to library entries of purified standards or recurrent unknown entities. Metabolon maintains a library based on authenticated standards that contains the retention time/index (RI), mass to charge ratio (m/z), and chromatographic data (including MS/MS spectral data) on all molecules present in the library. Furthermore, biochemical identifications are based on three criteria: retention index within a narrow RI window of the proposed identification, accurate mass match to the library +/−10 ppm, and the MS/MS forward and reverse scores between the experimental data and authentic standards. The MS/MS scores are based on a comparison of the ions present in the experimental spectrum to the ions present in the library spectrum. While there may be similarities between these molecules based on one of these factors, the use of all three data points can be utilised to distinguish and differentiate biochemicals. More than 3300 commercially available purified standard compounds have been acquired and registered into LIMS for analysis on all platforms for determination of their analytical characteristics. Additional mass spectral entries have been created for structurally unnamed biochemicals, which have been identified by virtue of their recurrent nature (both chromatographic and mass spectral). These compounds have the potential to be identified by future acquisition of a matching purified standard or by classical structural analysis.

### LC-MS for Amino Acid (AA) Quantification in Heart Tissues

Mouse heart tissues (10-30 mg) were weighted and minced into small pieces, to which 360 μL of ice-cold acetonitrile containing 0.1% formic acid and 50 μL of 25 UM ASPN-d3 and AA-13C4-15N mixture was added and then taken for homogenisation. The heart tissue was homogenised using a tissue-lyser for 1 min. The homogenised tissue extract was then centrifuged at 18,000 g for 40 minutes at 4° C. The supernatant was collected and again centrifuged at 18,000 g for another 30 minutes to remove any tissue fragments. 75 μL of the clear supernatant was transferred to a 96-microwell plate and loaded into the auto-sampler (7° C.) for analysis by LC-MS/MS. Ion counts were then normalised against that of the heavy isotope standard, before using the standard curve for quantification. AA calibration standards were prepared at concentrations 5000 UM, 2500 UM, 1250 UM, 625 UM, 312.5 μM, 156.25 μM, 78.125 μM, 39.0625 μM, 19.53125 μM, 9.765625 μM and 4.8828125 μM. Linearity for asparagine and aspartic acid was evaluated using the calibration curve generated with linear regression and 1/x weighting. Chromatographic separation was achieved on an Agilent 1290 Infinity II using a Supelco PEEK coated HPLC Column SeQuant® ZIC® cHILIC, 3 μm, 100 Å, 100×2.1 mm (Merck Pte Ltd, Singapore), set at a temperature of 40° C. Mobile phase A and B were 0.1% formic acid in water and 0.1% formic acid in acetonitrile, respectively. The chromatographic run time was set at 22 minutes, with a flow rate of 0.4 mL/min, and the maximum pressure was set at 450 bar. The elution gradient was set according to the experimental method, starting with 10% phase B at 0 mins with a linear increase to 70% at 11 mins, and back to 10% at 11.50 mins for column reconditioning. Flow was kept at 0.4 mL/min. Mass spectrometry quantification was performed using an Agilent 6495C, QQQ MS/MS detector with multiple reaction monitoring (MRM) and electrospray ionization in either positive or negative ionization mode. Source parameters were optimised and set according to the following, source gas temperature at 290° C., gas flow rate at 20 l/min, Nebulizer at 40 psi, sheath gas temperature at 400° C., sheath gas flow rate at 11.01 l/min. The sample injection volume was 3 μL. The parameters for the multiple reaction monitoring (MRM) used for asparagine and its corresponding heavy isotope standard are 131→114.1 with collision energy of 10 eV and Collision Accelerator Voltage (CAV) of 5V and 133.9→42.1 with collision energy of 10 eV and CAV of 5V, respectively with negative polarity. Similarly for aspartic acid and its corresponding heavy isotope standard are 132→88 with collision energy of 10 eV and CAV of 5V and 137→92 with collision energy of 10 eV and CAV of 5V, respectively with negative polarity. Acquisitions and quantitative analysis were performed using the Agilent MassHunter Workstation Acquisition (10.0.127) and Agilent MassHunter Workstation Quantitative Analysis (10.0) software.

### Bulk RNA Sequencing

Total RNA was carried through library preparation and subjected to Illumina sequencing. RNA integrity number (RIN) was measured with a Bioanalyzer 2100, using the Agilent RNA 6000 Pico kit, to ensure stringent RNA quality for samples before library preparation.

Total RNA sequencing libraries were generated with the Truseq Stranded Total RNA Library Prep kit, which involved the use of Ribo-Zero to remove abundant cytoplasmic rRNA. The remaining intact RNA was fragmented using a chemical mix, followed by first- and second-strand cDNA synthesis using random hexamer primers. “End-repaired” fragments were ligated with a unique Illumina adapter. All individually indexed samples were subsequently pooled together and multiplexed for sequencing. Libraries were sequenced using the Illumina Hiseq 4000 sequencing system and paired-end 150 bp reads were generated for analysis.

### Quality-Control (QC) and Bioinformatics Analysis of Bulk RNA Sequencing Data

We used established quality-control tools such as CASAVA version 1.8.2 (Illumina), FASTQC (Babraham Bioinformatics) and Trimmomatic to filter raw reads. Filtered reads were aligned to mouse genome (Mus musculus) mm9 assembly using mapping software, STAR version 2.5.3 using default settings. Gene count per sample was computed with htseq-count, using mm9 GENCODE version M1. Differential expression analysis was performed using either R package edgeR or DESeq2 with statistical significance cut-off as FDR <0.05, fold-change >2 (for OSKM-CMs v.s. Control-CMs) or padj<0.05, fold-change>1.5 (OSKM1×Asns^{OE} v.s. OSKM1×BFP^{OE}). PCA was performed using the PCA function in R package FactoMineR. The Homer Motif Analysis was used for motif enrichment analysis. GO enrichment analysis was performed by enrichGO function in R package clusterProfiler.

### Weighted Gene Correlation Network Analysis (WGCNA)

Using WGCNA, we started the construction of a signed weighted correlation network by computing pairwise correlations between all genes across all RNA sequencing samples. Next, we chose soft thresholding power (β=6), in constructing an adjacency matrix using the formula, aij=(0.5+0.5×sij)β, where aij is defined as weighted correlation and sij is defined coefficient correlation between genei and genej. We chose the power (β=6), which is the lowest power for which the scale-free topology fit index curve flattens out upon reaching a high value of 0.98. Using the adjacency matrix computed in the previous step, topological overlap was calculated to measure the network interconnectedness in a robust and biological meaningful way. The topological overlap was utilised to group highly correlated genes together using average linkage hierarchical clustering. Modules were defined as the branches obtained by cutting the hierarchal tree using Dynamic Hybrid Tree Cut algorithm. We defined the first principle component of a module as module eigengene, which is representative of the expression profile in each module. Genes in each module were removed if the correlation between the gene and module eigengene (kME) was <0.3. If a detected module did not have at least 5 genes with eigengene connectivity (kME) at least 0.5, the module was disbanded and its genes were unlabeled and returned to the pool of genes to await module detection. Modules whose eigengenes were highly correlated (correlation above 0.75) were merged. Construction of signed gene network and identification of modules were performed using R function, blockwiseModules with the following parameters: soft thresholding power=3, minimum module size=15, mergeCutHeight=0.25, corType=“Pearson”, networkType=“signed”, TOMType=“signed”, minCoreKME=0.5, and minKMEtoStay=0.3.

### Quantification and Statistics

Data were presented as mean±SD. Two-tailed, unpaired Student's t test was performed for comparisons between two groups. One-way ANOVA followed by multiple comparison test was used to compare more than two groups. Two-way ANOVA followed by multiple comparison test was used to compare more than two groups across a specific experimental timeline (the two factors being time and different treatment, respectively). Chi-square test was used when comparing the frequencies in one or more categories of a contingency table. Logrank test was used to compare survival curves between two or more groups. Details of tests and comparisons were listed in the figure captions. All tests were performed using GraphPad Prism 8 software. p<0.05 was considered significant.

TABLE 1
Materials used in the present application

| | Source | Catalogue No. | Dilution |
|---|---|---|---|
| Primary antibodies | | | |
| Mouse-anti-α-Actinin (EA-53) | Sigma-Aldrich | Cat#: A7811 | 1:1000 (IF) |
| Rabbit-anti-MYBPC3 | Abcam | Cat#: ab171153 | 1:200 (IF) |
| Goat-anti-cTnl | Abcam | Cat#: ab56357 | 1:500 (IF) |
| Rabbit-anti-OCT3/4 (H-134) | Santa Cruz | Cat#: sc-9081 | 1:400 (WB) |
| | Biotechnology | | 1:1000 (IF) |
| Mouse-anti-OCT3/4 (C-10) | Santa Cruz | Cat#: sc-5279 | 1:100 (IF) |
| | Biotechnology | | |
| Rabbit-anti-SOX2 | Abcam | Cat#: ab97959 | 1:1000 |
| | | | (WB) |
| Rabbit-anti-SOX2 | Proteintech | Cat#: 20118-1- | 1:200 (IF) |
| | | AP | |
| Rabbit-anti-HA Tag | Abcam | Cat#: ab9110 | 1:1000 |
| | | | (WB) |
| Rabbit-anti-V5 Tag | Abcam | Cat#: ab9116 | 1:1000 |
| | | | (WB) |
| Mouse-anti-GAPDH (GA1R) | Thermo Fisher | Cat#: MA5-15738 | 1:2000 |
| | | | (WB) |
| Rabbit-anti-αSMA | Abcam | Cat#: ab5694 | 1:200 (IF) |
| Mouse-anti- αSMA | Sigma-Aldrich | Cat#: A5228 | 1:20,000 |
| | | | (IF) |
| Mouse-anti-Connexin 43 (F-7) | Santa Cruz | Cat#: sc-271837 | 1:500 (IF) |
| | Biotechnology | | |
| Rabbit-anti-Ki67 | Abcam | Cat#: ab15580 | 1:1000 (IF) |
| Rat-anti-Ki67 | Thermo Fisher | Cat#: 14-5698-82 | 1:200 (IF) |
| Rabbit-anti-pH3 | Merck Millipore | Cat#: 06-570 | 1:250 (IF) |
| Rabbit-anti-Aurora kinase B | Abcam | Cat#: ab2254 | 1:250 (IF) |
| Mouse-anti-ASNS (G-10) | Santa Cruz | Cat#: sc-365809 | 1:1000 |
| | Biotechnology | | (WB) |
| Rabbit-anti-ASNS | Proteintech | Cat#: 14681-1- | 1:100 (IF) |
| | | AP | |
| Chicken-anti-GFP | Abcam | Cat#: ab13970 | 1:1000 (IF) |
| Rabbit-anti-RFP | Rockland | Cat#: 600-401- | 1:500 (IF) |
| | | 379 | |
| Biotinylated Wheat Germ | Vector Labs | Cat#: B-1025 | 1:100 (IF) |
| Agglutinin (WGA) | | | |
| Rabbit-anti-phosphorylated | Cell Signaling | Cat#: 9205 | 1:1000 |
| S6K1 (Thr389) | Technology | | (WB) |
| Rabbit-anti-S6K1 | Cell Signaling | Cat#: 9202 | 1:1000 |
| | Technology | | (WB) |
| Rabbit-anti-phosphorylated | Cell Signaling | Cat#: 2855 | 1:2000 |
| 4EBP1 (Thr37/46) (236B4) | Technology | | (WB) |
| Rabbit-anti-4EBP (53H11) | Cell Signaling | Cat#: 9644 | 1:2000 |
| | Technology | | (WB) |
| Mouse-anti-GLUL | Santa Cruz | Cat#: sc-74430 | 1:1000 |
| | Biotechnology | | (WB) |
| Secondary antibodies | | | |
| Goat anti-Mouse, Alexa Fluor | Thermo Fisher | Cat#: A-11001 | 1:1000 (IF) |
| 488 | | | |
| Goat anti-Mouse, Alexa Fluor | Thermo Fisher | Cat#: A-11005 | 1:1000 (IF) |
| 594 | | | |
| Goat anti-Rabbit, Alexa Fluor | Thermo Fisher | Cat#: A-11008 | 1:1000 (IF) |
| 488 | | | |
| Goat anti-Rabbit, Alexa Fluor | Thermo Fisher | Cat#: A-11012 | 1:1000 (IF) |
| 594 | | | |
| Goat anti-Chicken, Alexa Fluor | Thermo Fisher | Cat#: A-11039 | 1:1000 (IF) |
| 594 | | | |
| Donkey anti-goat, Alexa Fluor | Thermo Fisher | Cat#: A-21447 | 1:1000 (IF) |
| 647 | | | |
| Donkey anti-rat, Alexa Fluor | Thermo Fisher | Cat#: A-21208 | 1:1000 (IF) |
| 488 | | | |
| Stretavidin linked fluorophore, | Thermo Fisher | Cat#: S11223 | 1:500 (IF) |
| Alexa 488 and 568 | | Cat#: S11226 | |

| | Source | Catalogue No. |
|---|---|---|
| Plasmids | | |
| pGL4. 10[luc2] vector | Promega | Cat#: E6651 |
| pGL4.73[hRluc/SV40] vector | Promega | Cat#: E6911 |
| pGL4.13[luc2/SV40] vector | Promega | Cat#: E6681 |
| Critical commercial assays | | |
| qScript cDNA Synthesis Kit | Quantabio | Cat#: 95047 |
| Perfecta Sybr Green low ROX | Quantabio | Cat#: 95074 |
| Protein assay kit I | Bio-Rad | Cat#: 5000001 |
| WESTAR Supernova ECL | Cyanagen | Cat#: XLS3, 0100 |
| jetPRIME ® transfection kit | PolyPlus | Cat#: 114-75 |
| Dual-Glo Luciferase Assay System | Promega | Cat#: E2940 |
| Agilent RNA 6000 PicoS kit | Agilent | Cat#: 5067-1513 |
| Truseq Stranded Total RNA Library Prep | Illumina | Cat#: RS-122-2201 |
| kit | | |

| Cell line | Source | Accession No. |
|---|---|---|
| HEK293T | ATCC | CRL-3216 |

### Example 2

### Adult Mouse CMs Lost Maturity Through CMDD by In Vitro Culture

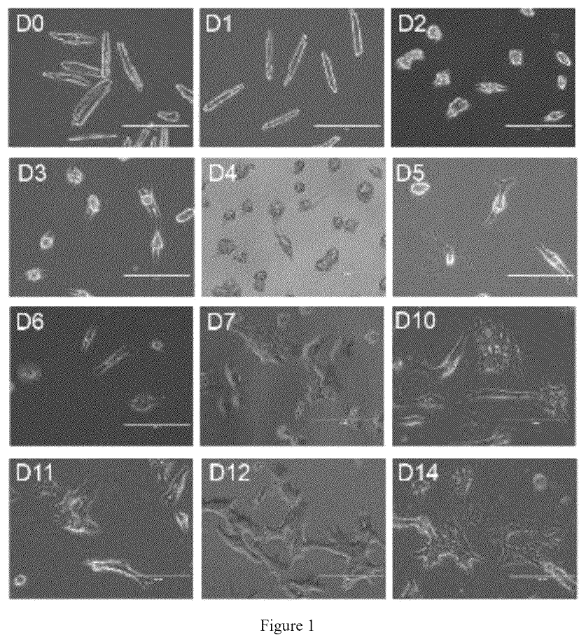

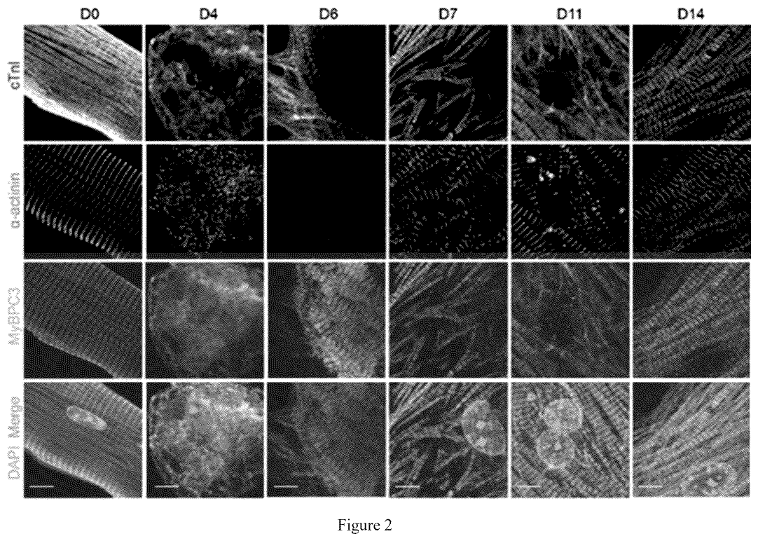

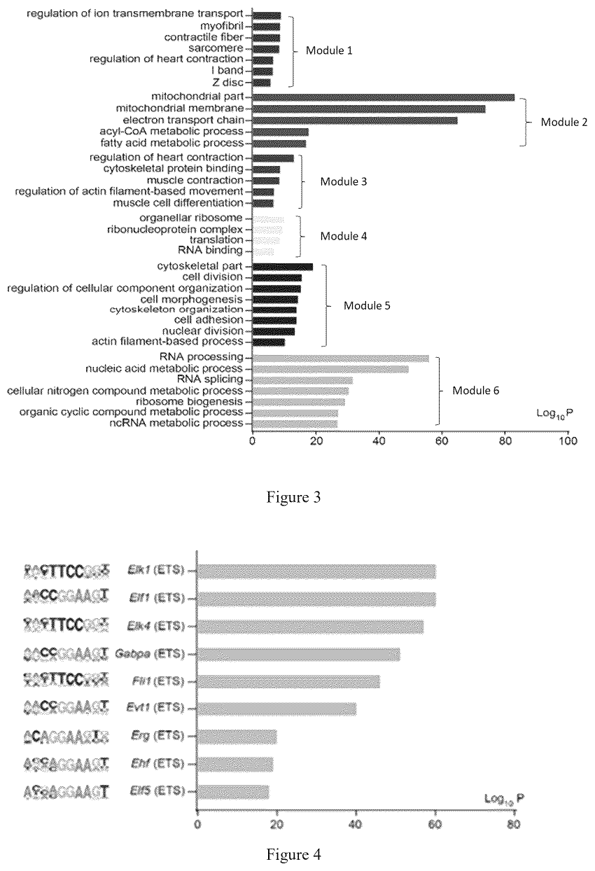

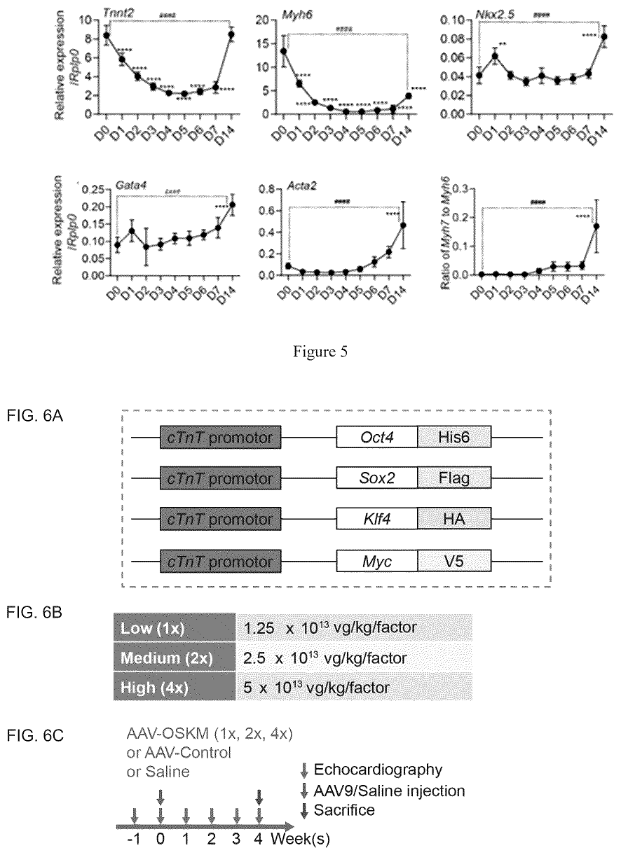

Adult mouse CMs (ACMs) were isolated and purified using a protocol previously published (See Ackers-Johnson et al., 2016), and cultured for 14 days. Phenotypic CMDD progressed from D0 to D7, followed by partial re-differentiation from D7 to D14, indicated by the breakdown and re-construction of sarcomeres (FIGS. 1-2). RNA-seq and weighted gene correlation network analysis (WGCNA) were performed to explore transcriptomic changes from D0 to D14. Genes chronologically downregulated and re-upregulated (Modules 1, 2 and 3) were related to heart contraction, sarcomeres, myofibril assembly, regulation of ion transport and fatty acid metabolism. Conversely, genes upregulated and then downregulated (Modules 4, 5 and 6) were enriched for organellar ribosome, nucleic acid metabolism and mitotic cell cycle (FIG. 3), all correlating to CMDD and re-differentiation processes. HOMER motif analysis revealed the gene module 6 to be enriched for the binding motif of ETS family (FIG. 4), which regulates Gata4 enhancers during embryonic cardiac development (Schachterle et al., 2012). Some early cardiac genes Acta2, Nkx2.5, Gata4, Myh7/Myh6 ratio were also upregulated significantly at D14, reminiscent of mouse heart development (FIG. 5). These results indicate that in vitro cultured ACMs lost maturity undergoing CMDD with subsequent transition to re-differentiation albeit without detectable proliferation.

### Example 3

### CM-Specific Overexpression of OSKM by AAV9 Induced Loss of Cardiac Function and Hallmarks of CMDD In Vivo

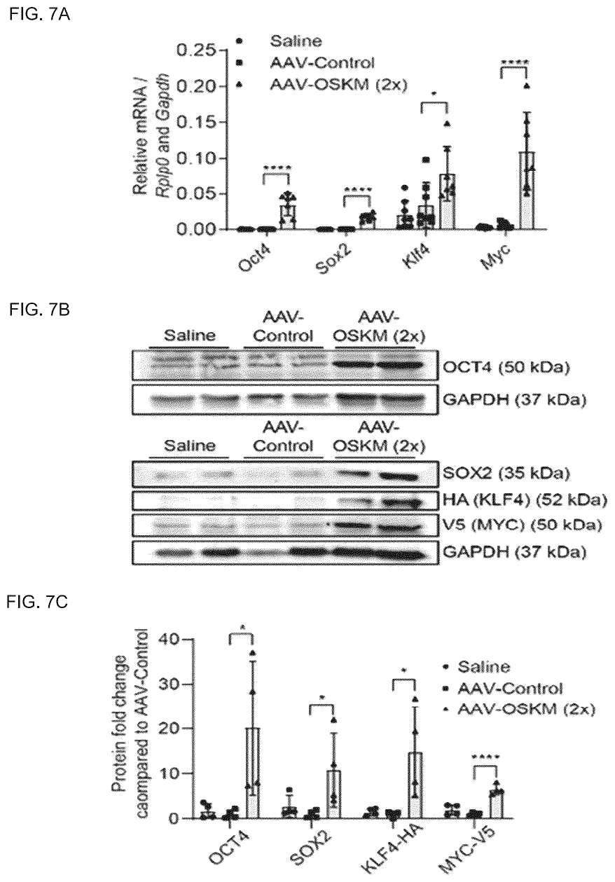

Genetic or adenovirus-based induction of OSKM has been reported to result in partial reprogramming and CMDD in vivo. A cocktail comprising four AAV9 (AAV-OSKM) was injected into adult mice at three doses (FIGS. 6A-6C), and validated CM-specific OSKM overexpression (FIGS. 7A-7C). At low dose (1×), no echocardiographic or histological changes were detected within 4 weeks, while high dose (4×) led to a precipitous loss of cardiac function, myocardial fibrosis and premature death by D21. Mice receiving the mid dose (2×) exhibited qualitatively similar, but milder phenotypes and a longer survival than those receiving high dose. AAV-OSKM (2×) was therefore used for further experiments (termed “OSKM group” hereafter).

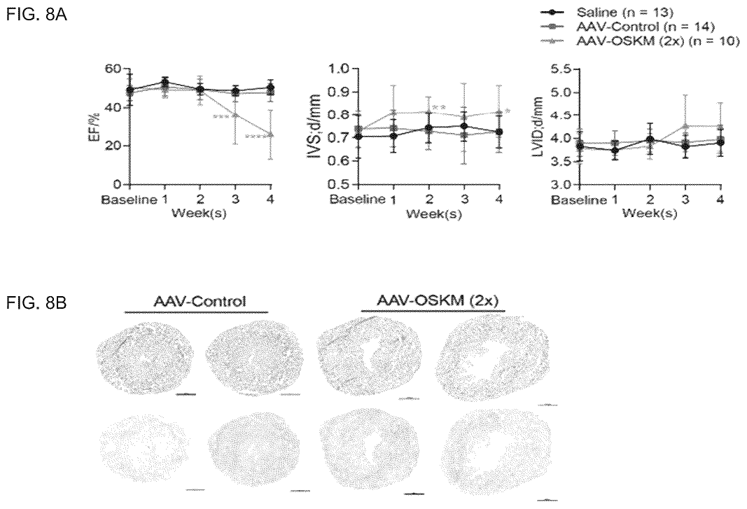

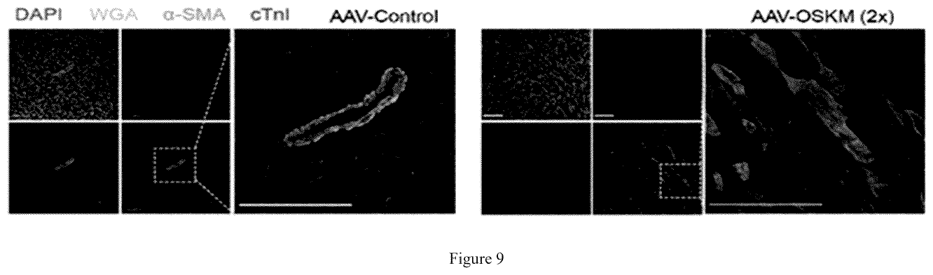

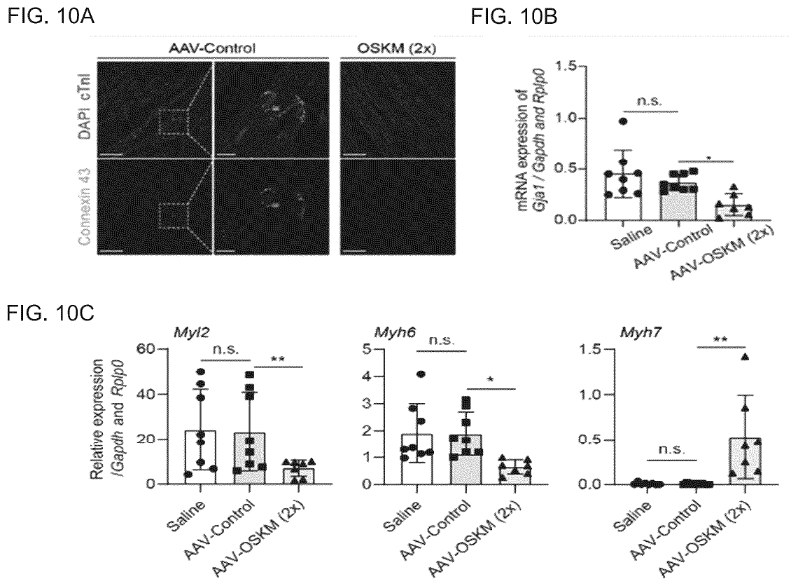

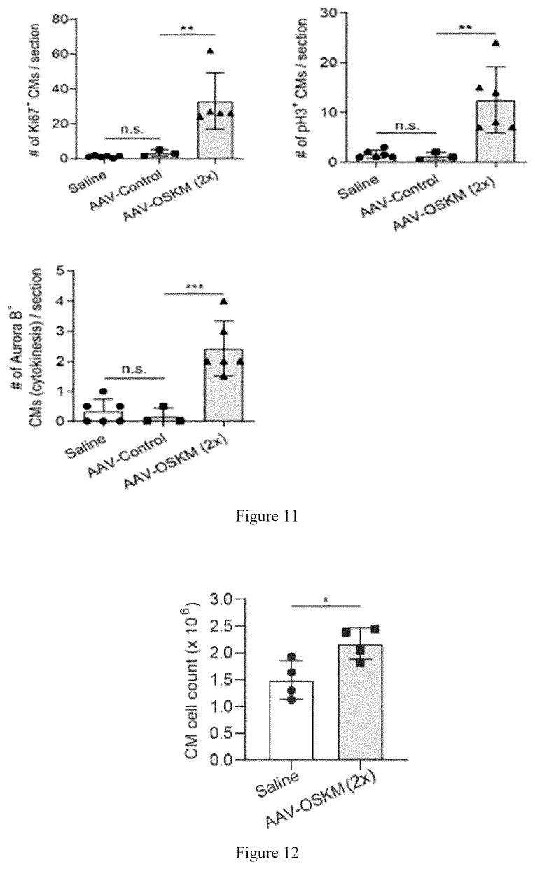

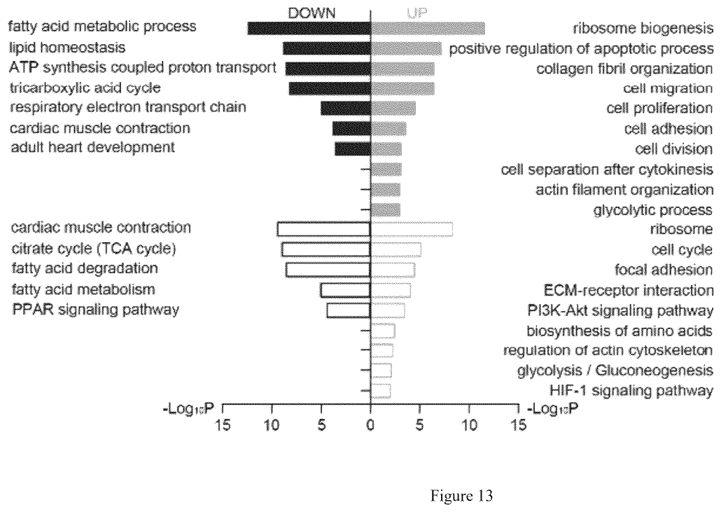

Four weeks post AAV injection, the OSKM group showed reduced cardiac function, increased ventricular wall thickness and increased heart weight (FIGS. 8A-8B), consistent with prolonged CMDD producing the loss of vital cellular function. AAV-OSKM (2×) led to higher CM aSMA (Acta2) expression (FIG. 9), diminished CM junction protein Connexin 43 (Gja1), lower sarcomeric gene expression (FIGS. 10A-10C), and more CMs positive for cell cycle (Ki67), mitosis (pH3) and cytokinesis (Aurora kinase B) markers (FIG. 11). The absolute CM count was also higher in OSKM group (FIG. 12), indicating CM proliferation as previously published. RNA-seq confirmed that OSKM-CMs upregulated ribosome biogenesis, glycolysis, and downregulated fatty acid metabolism, TAC cycle, mitochondrial electron transport and muscle contraction pathways (FIG. 13). These results reflect that AAV9-mediated OSKM induction resulted in partial CM reprogramming and CMDD in vivo, faithfully matching the hallmarks previously described.

### Example 4

### Integrative Analysis of RNA-Seq Datasets Identified Asparagine Synthetase (Asns) as a CMDD Associated Marker Gene

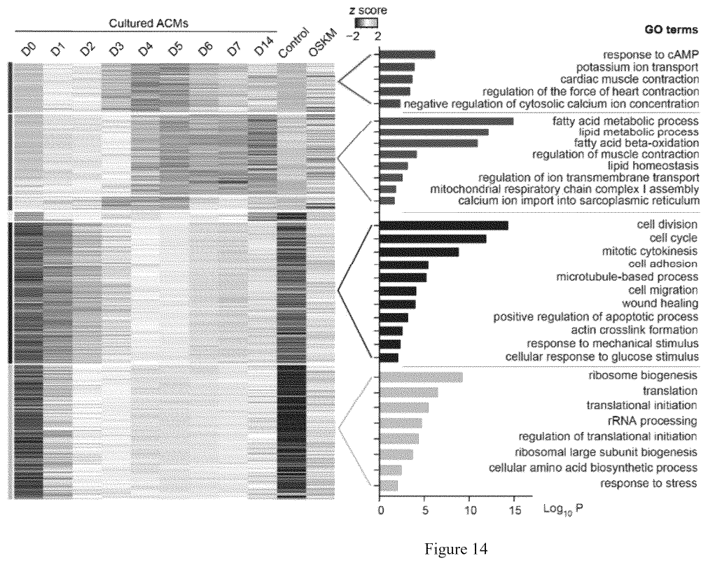

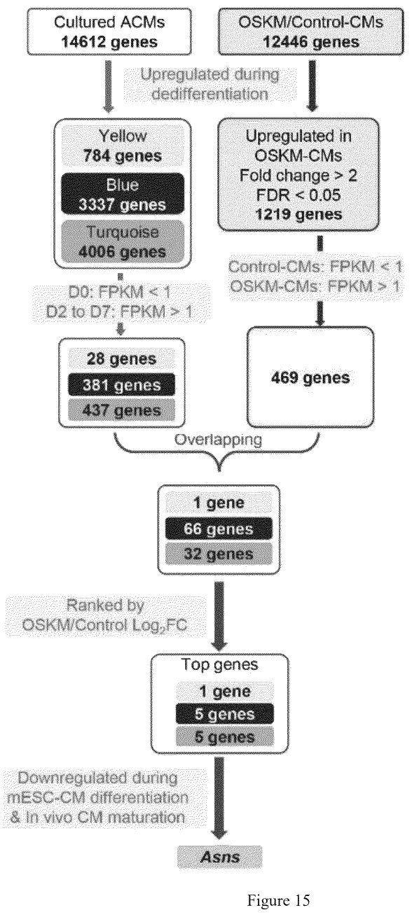

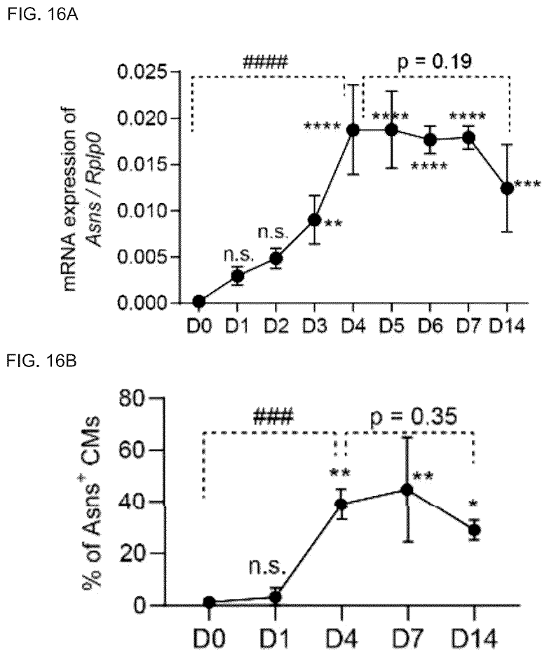

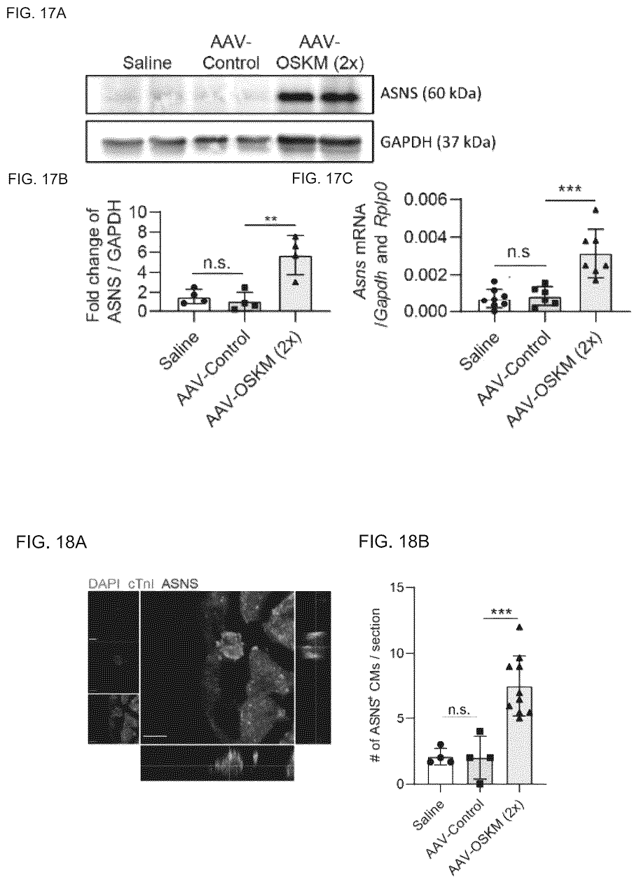

Next, principal component analysis (PCA) of the transcriptomes was performed, which effectively separated OSKM-CMs from Control-CMs, uncovering a clear trajectory of evolving transcription in cultured ACMs from D0 to D14. Interestingly, D14-ACMs clustered adjacent to OSKM-CMs, consistent with CMDD in OSKM and underpinning the expectation of a common dominator from the transcriptomes of both models. In both cultured ACMs (D0 to D7) and OSKM-CMs, downregulated genes were enriched for ion transport, muscle contraction and fatty acid metabolism, while those upregulated were enriched for ribosome biogenesis, amino acid synthesis, translational initiation and cell cycling (FIG. 14). In follow up, CMDD marker candidates were isolated and ranked based on the following criteria: (i) upregulated in cultured ACMs (yellow, blue, turquoise modules) and OSKM-CMs; (ii) high abundance in dedifferentiated CMs; (iii) very low expression at baseline in normal CMs (detailed cut-offs shown in FIG. 15). The top-ranked genes were further shortlisted to those that are downregulated during mouse embryonic stem cell (mESC)-CM differentiation and in vivo CM maturation (from neonate to adult). Asns was the only candidate screened out (FIG. 15). Indeed, it is validated that Asns was virtually absent at baseline, and upregulated from D0 to D4, maintaining high expression from D4 to D14 in cultured ACMs (FIG. 16A). IF staining confirmed the same for ASNS-positive (ASNS⁺) CMs from D0 to D14 (FIG. 16B). Likewise, adult CMs did not express Asns, whereas OSKM-CMs significantly upregulated Asns for 4 to 5 folds, assessed by both RT-qPCR and Western blot (FIGS. 17A-17C). In OSKM histological sections, ASNS expression was validated in cTnl⁺ CMs (FIGS. 18A-18B) and was upregulated by 3 folds. Hence, starting at the baseline of zero, Asns expression upregulated significantly, and correlated well with progressive CMDD in both models.

Crucially, Asns expression was also assessed in other published datasets of transcriptomes involving CMDD or heart regeneration, alongside the two previously proposed CMDD markers, Dab2 and Runx1 (Kubin et al., 2011). Asns and Runx1 were upregulated in regenerative ErbB2-OE hearts, implying a crosstalk with Hippo-Yap signalling (See Aharonov et al., 2020). Asns was upregulated in Hippo-deficient pressure-overload mouse model with prolonged CMDD (See Ikeda et al., 2019), but not in Hippo-deficient CMs post myocardial infarction (MI) (See Leach et al., 2017). As described above, Asns was downregulated during mESC-CM differentiation and in vivo maturation, while Dab2 and Runx1 were not (See O'Meara et al., 2015). Single cell RNA-seq revealed more Asns-expressing CMs in regenerating P1 mouse heart post MI (See Cui et al., 2020; Quaife-Ryan et al., 2017). Furthermore, in the FUCCI tracking system, ASNS was higher in active cycling human CMs, but not in cell cycle-arrested ones (See Mohamed et al., 2018). Besides mouse and human, Asns was also upregulated in regenerating hearts of zebrafish (See Ben-Yair et al., 2019) and P2 pigs post injury (See Ye et al., 2018). Collectively, Asns shows consistent potential as a CMDD marker, even in at least two other species.

### Example 5

### The Subpopulation of ASNS-Positive CMs Displayed Key CMDD Features

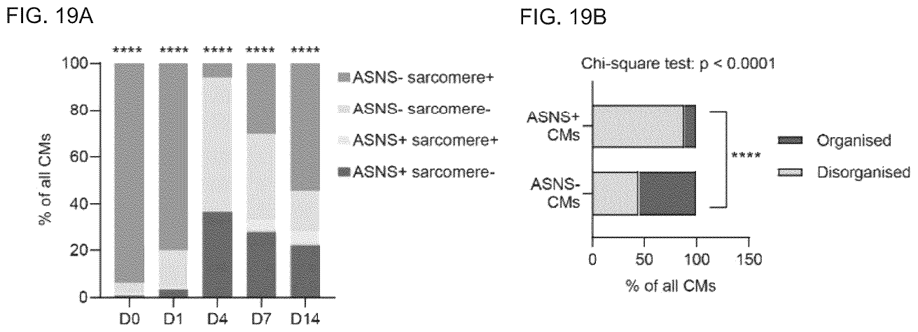

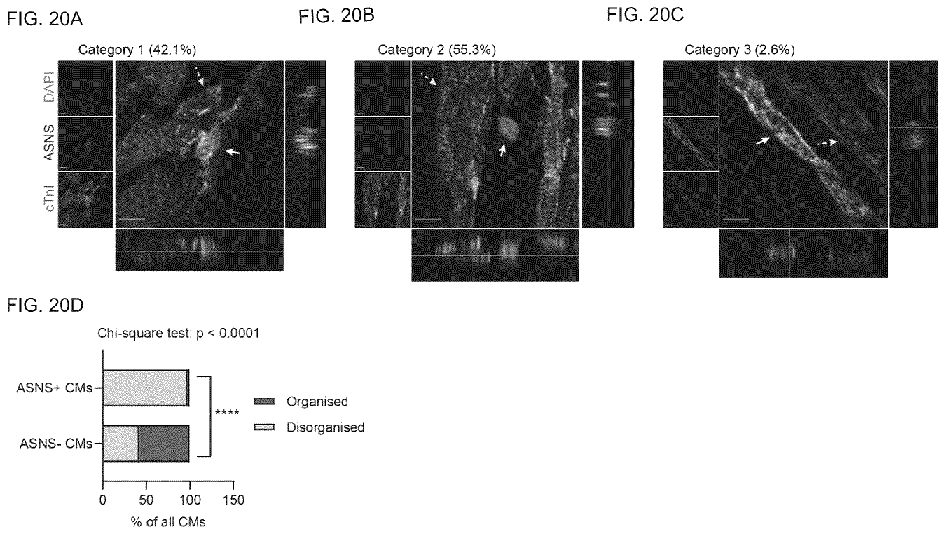

IF staining demonstrated that not all cultured ACMs and OSKM-CMs were unequivocally positive for ASNS (FIGS. 16B, 18B), which may imply that they do not dedifferentiate synchronously, or that some CM do not cross the Asns expression threshold. Hence, ASNS⁺ and ASNS-negative (ASNS⁻) CMs were characterised and compared, by taking snapshots of cultured ACMs from D0 to D14, classifying and quantifying them into four categories based on their sarcomeric organisation and ASNS expression (FIG. 19A). ASNS⁺ CMs exhibited a higher proportion of disorganised sarcomeres compared to ASNS⁻ CMs, either by analysing individual timepoints (FIG. 19A) or collectively for all D0 to D14 ACMs (FIG. 19B). Similarly, OSKM-CMs were categorised by the confocal microscopy images for both ASNS⁺ and ASNS⁻ CMs (FIG. 20A). Most images (55.3%) fell into Category 2 (Cat #2), where ASNS⁻ CMs exhibited discernible sarcomeres, and neighbouring ASNS⁺ CM(s) did not (FIG. 20B). Cat #3 comprised only 2.6% of the images, where both ASNS⁻ and ASNS⁺ CMs exhibited obvious sarcomeres (FIG. 20C). In Cat #1, although both subpopulations lacked discernible sarcomeres (FIG. 20A), this may be attributed to the transverse sectioning angle (See Tskhovrebova and Trinick, 2012). In this case, by pooling up Cat #2 and #3, ASNS⁺ CMs still showed a significantly higher proportion with disorganised sarcomeres (FIG. 20D).

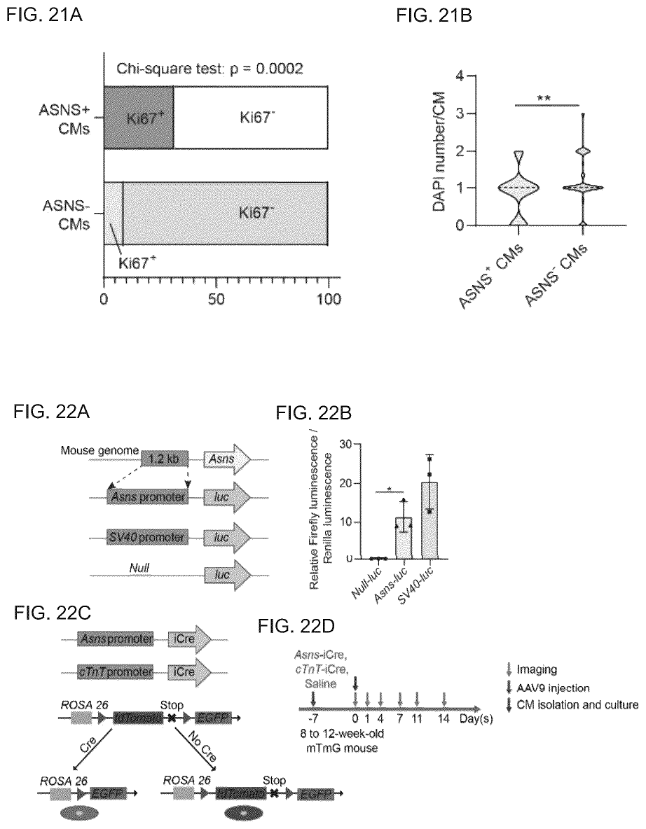

Additionally, co-staining and quantification of ASNS and Ki67 on OSKM-heart sections revealed that ASNS⁺ CMs showed higher proportion positive for Ki67 (Ki67⁺) (FIG. 21A), indicating a higher proliferative potential, compared to ASNS⁻ CMs. It is also reported that after division, daughter CMs are mostly mononucleated, while pre-existing CMs are mostly binucleated or multinucleated. Consistent with this, the majority of ASNS⁺ CMs exhibited only 1 DAPI signal, compared to ASNS⁻ CMs (FIG. 21B), supporting the correlation between OSKM-induced CM cell cycle and ASNS expression. Collectively, in both models, ASNS⁺ CMs displayed distinct features of CMDD, compared to ASNS⁻ CMs.

### Example 6

### Asns-Promoter Driven Cre Recombinase Enabled Tracking of CMDD Process

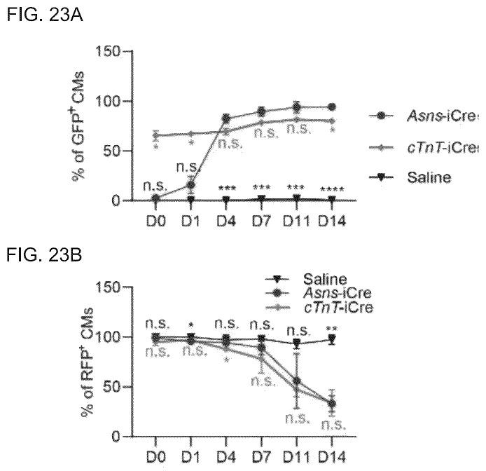

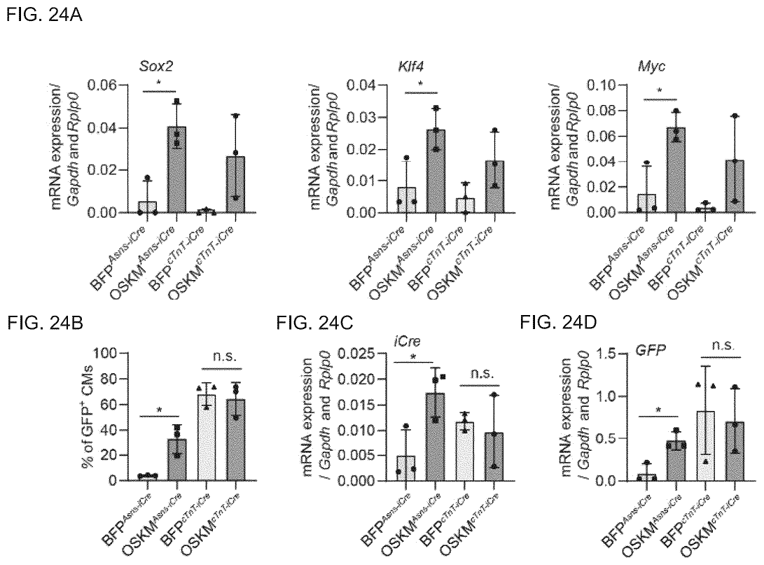

To harness Asns for tracking CMDD, the mouse genomic sequence of Asns (GEO accession No: GSE31039) was analysed and a total fragment of 1.2 kb was used as core Asns promoter, capable of driving minimum luciferase expression in HEK293T cells (FIGS. 22A-22B). With this, AAV9 comprising an Asns-promoter driven improved-Cre recombinase (See Shimshek et al., 2002) (AAV-Asns-iCre) was constructed and injected into adult mTmG^{+/-} mice (See Muzumdar et al., 2007), where activated iCre triggers the switch from tdTomato to GFP expression (FIG. 22C). Adult mTmG^{+/-} mice were also injected with saline or AAV-cTnT-iCre, which were used as negative control and positive control respectively. One week post AAV9 injection, CMs were isolated and cultured for another 14 days as already described (FIG. 22D). As expected, throughout the culture, ACM^{Saline} (negative control) and ACM^{cTnT-iCre} (positive control) displayed minimal versus considerable GFP⁺ CMs, respectively. Notably, ACM^{Asns-iCre} showed very low percentage of GFP⁺ CMs on D0, which increased to >80% from D4 until D14, showing progressive GFP expression concurrent with the initiation of CMDD, as tracked by the activated Asns promoter (FIG. 23A). Surprisingly, tdTomato and GFP co-existed in some ACMs, with tdTomato⁺ CMs decreasing only after D7 (FIG. 23B). This is attributed to a long half-life of tdTomato. The same hypothesis was tested in OSKM-CMs in vivo by injecting a mixture of AAV-Asns-iCre and AAV-OSKM (2×) into adult mTmG^{+/-} mice (OSKM^{Asns-iCre}), with AAV-Asns-iCre and AAV-cTnT-BFP (BFP^{Asns-iCre}) injected as negative control. Four weeks after AAV injection, RT-qPCR confirmed high expression of Sox2, Klf4, Myc in OSKM^{Asns-iCre} hearts. Meanwhile, the OSKM^{Asns-icre} group showed significantly more GFP⁺ CMs (FIG. 24A-B), with higher iCre and GFP mRNA expression (FIGS. 24C-24D) than BFP^{Asns-iCre}. As positive controls, both OSKM^{cInT-iCre} and BFP^{cInT-iCre} groups displayed prevalent GFP⁺ CMs (FIG. 24B). Taken together, Asns-promoter driven iCre, delivered by AAV9, enabled an efficient tracking of CMs as they dedifferentiated both in vitro and in vivo.

### Example 7

### Asns is an Essential Mediator of CMDD Through the Regulation of Asparagine Metabolism

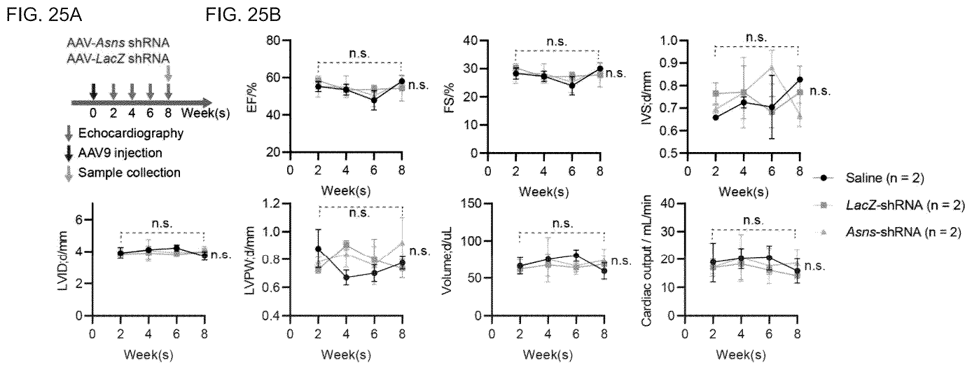

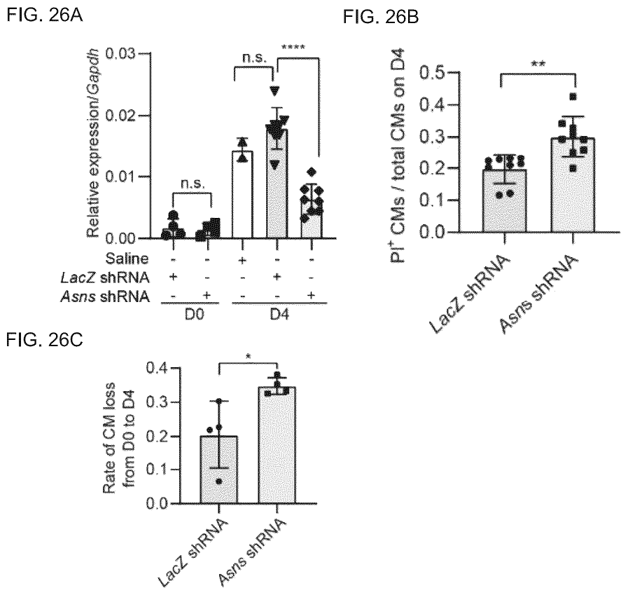

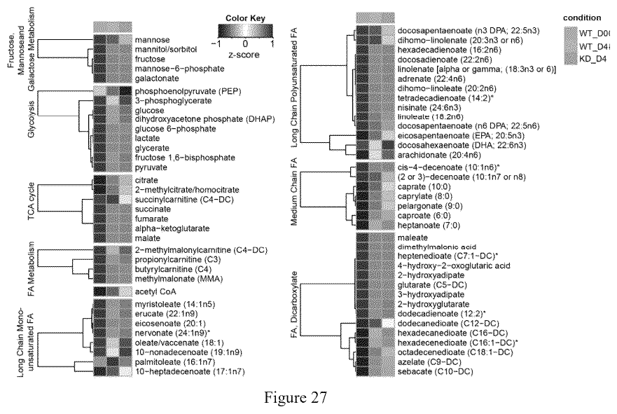

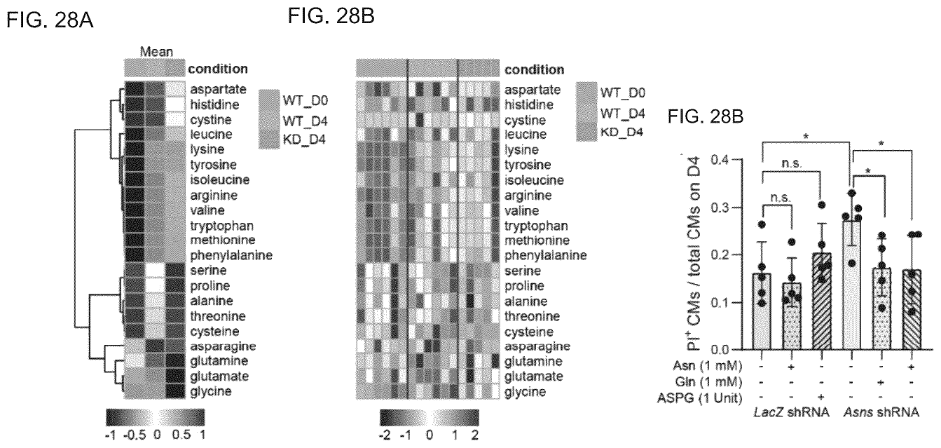

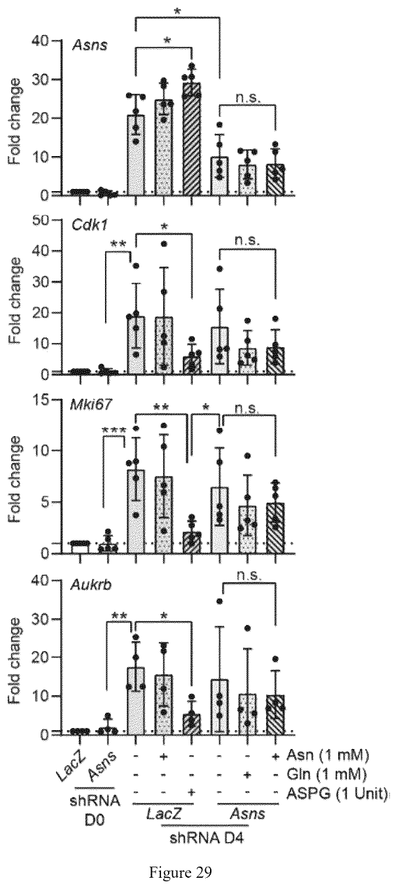

To test whether Asns regulates CMDD, Asns was knocked down (Asns^{KD}) with shRNA using AAV9-cTnT-Asns shRNA-GRP. Asns^{KD} did not affect myocardial function or structure up to 8 weeks in vivo (FIGS. 25A-25B). KD efficiency was validated in isolated and cultured CMs 1 week after AAV9 injection (FIGS. 26A-26C). Compared to LacZ^{KD} control, Asns^{KD} resulted in increased CM death on D4 but not on D0, quantified by propidium iodide (PI) (FIGS. 26B-26C). Given the potential vital role of Asns in amino acid homeostasis, an untargeted metabolomics study of cultured ACMs was performed. Major energy-related metabolic pathways, such as glycolysis, TCA cycle and fatty acid oxidation were largely unaltered by Asns^{KD} on D4 (FIG. 27). Instead, an overall increase in subsets of amino acids (histidine, cystine, leucine, isoleucine, valine, lysine, tyrosine, and others) during CMDD, and a particular subset (serine, proline, alanine, glycine, and glutamate) perturbed by Asns^{KD} was observed. Among them, asparagine (Asn) and glutamine (Gln), two key components in ASNS catalysis, were significantly upregulated during CMDD and abrogated by Asns^{KD} on D4 (FIGS. 28A-28B). Treating cultured ACMs with either Asn or Gln rescued Asns^{KD}-induced CM death (FIGS. 28A-28B). Interestingly, eliminating Asn by L-asparaginase (ASPG) did not induce more cell death, but diminished the expression of cell cycle genes (FIG. 29), indicating that ASPG alone is insufficient to phenocopy the effect of Asns deficiency.

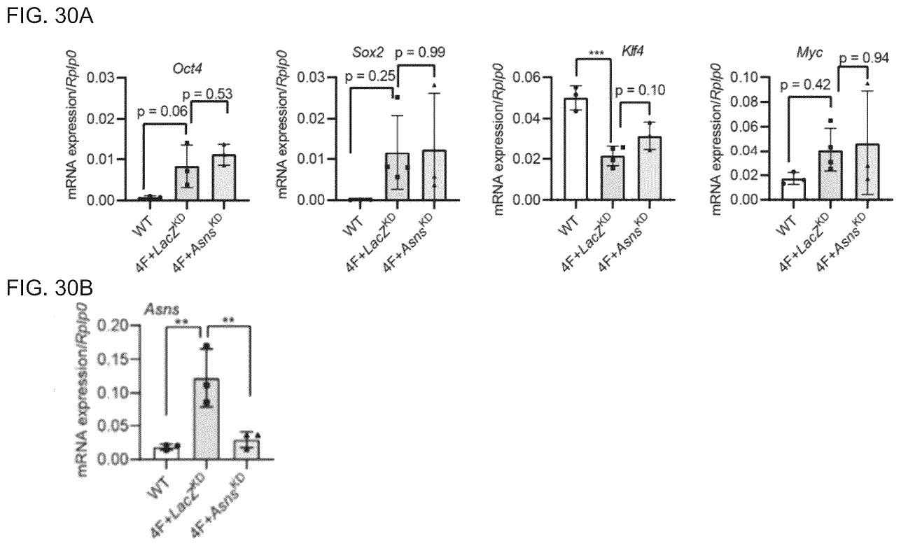

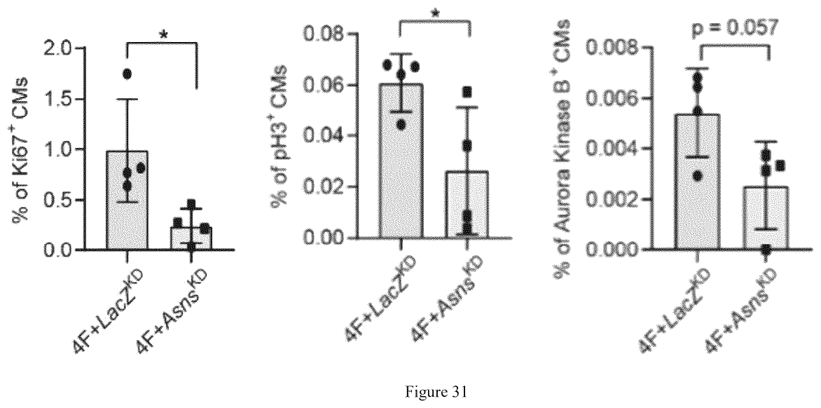

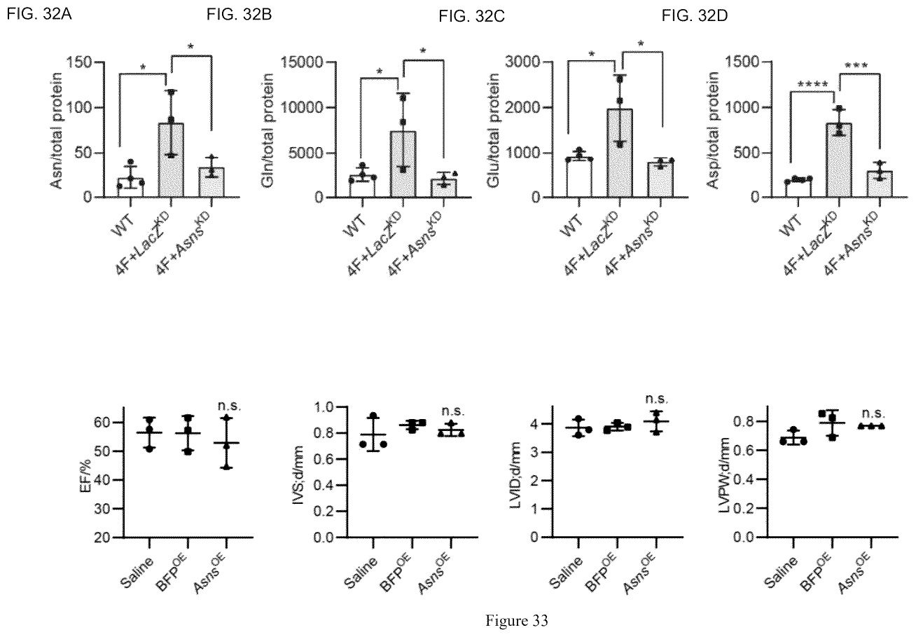

Next, AAV-cTnT-Asns-shRNA-GFP was injected to knock-down Asns in vivo in the recently reported “i4FHeart” mouse, which overexpresses CM-specific OSKM upon doxycycline induction (See Chen et al., 2021). Asns^{KD} was efficient without affecting OSKM overexpression levels (FIGS. 30A-30B). However, unlike Asns^{KD} in cultured ACMs, Asns^{KD} (4F+Asns^{KD}) in vivo here did not significantly alter CM death indices, compared to 4F+LacZ^{KD} group (data not shown). Instead, the 4F+Asns^{KD} group exhibited significantly fewer CMs staining for cell cycle markers Ki67, pH3 and Aurora kinase B (FIG. 31). Like the finding in vitro, LC-MS revealed that the four amino acids involved in ASNS catalysis-Asn, Gln, glutamate (Glu) and aspartate (Asp) were significantly upregulated by OSKM, and abrogated by Asns^{KD} (FIGS. 32A-32D). Hence, during CMDD, Asns appears upregulated as an essential gene for ACM survival undergoing CMDD in culture, and OSKM-induced CM cell cycle in vivo, at least in part by regulating Asn metabolism.

### Example 8

### Overexpression of Asns Augmented the Hallmarks of CMDD Induced by OSKM

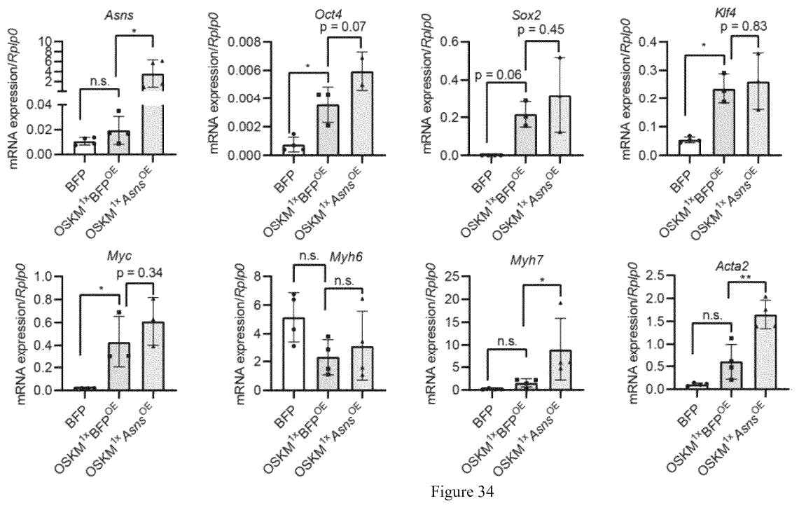

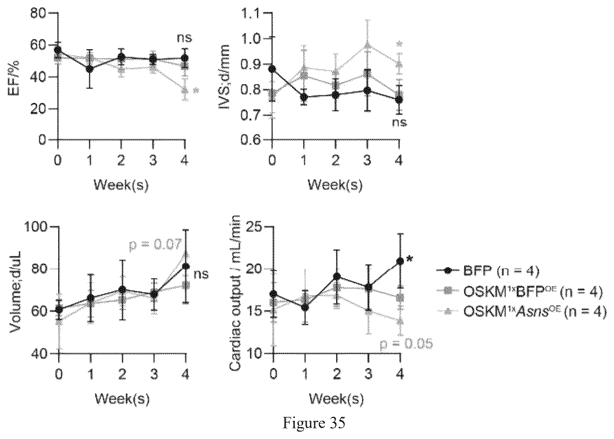

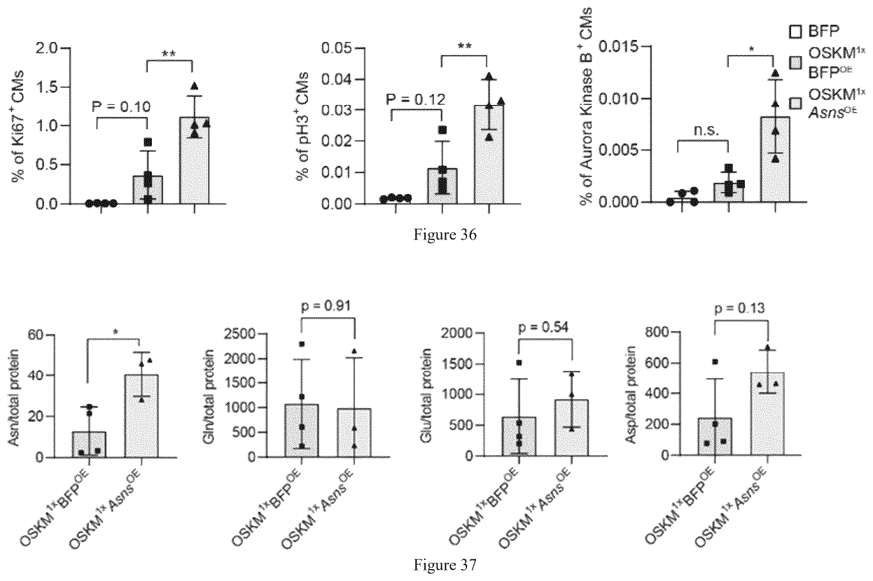

In gain-of-function by overexpressing CM-specific Asns using AAV-cTnT-Asns-V5, Asns^{OE} alone in adult healthy mice did not induce any detectable changes on echocardiography (FIG. 33). Instead, when low-dose AAV9-OSKM (1×) was injected together with Asns^{OE} (OSKM1×Asns^{OE}) in adult mice, with AAV-cTnT-BFP (BFP^{OE}) as control (OSKM1×BFP^{OE}), Asns^{OE} did not affect OSKM overexpression levels (FIG. 34), but OSKM1×Asns^{OE} group showed reduced ejection fraction (EF %) and increased interventricular wall thickness (IVS; d), resembling the compromised cardiac phenotype induced by OSKM (2×) (FIG. 35). In contrast, OSKM1×BFP^{OE} control mice did not show any change in cardiac function 4 weeks after injection. Importantly, compared to OSKM1×BFP^{OE}, OSKM1×Asns^{OE} showed significant CMs bearing cell cycle markers Ki67, pH3 and Aurora kinase B (FIG. 36). Similarly, OSKM1×Asns^{OE} showed upregulated remodelling markers α-SMA (Acta2) and Myh7 (FIG. 34). Bulk RNA-seq of heart tissues further revealed global downregulation of genes enriched for CM differentiation, muscle development and sarcomere components, and upregulated genes related to extracellular matrix organisation, tissue remodelling, ERK1/2 cascade in OSKM1×Asns^{OE} group (data not shown). In this context, only Asn was upregulated among the four amino acids measured by LC-MS, in OSKM1×Asns^{OE} hearts (FIG. 37). In summary, forced Asns^{OE} significantly augmented key hallmarks of CMDD, not on its own, but in association with OSKM. Asn is the predominant amino acid accumulated with these hallmarks, but it is also possible that Asns^{OE} mediates changes through other means in addition to Asn abundance.

### Example 9

Asns is Necessary for Sustaining Mammalian Target of Rapamycin (mTOR) in Dedifferentiating CMs

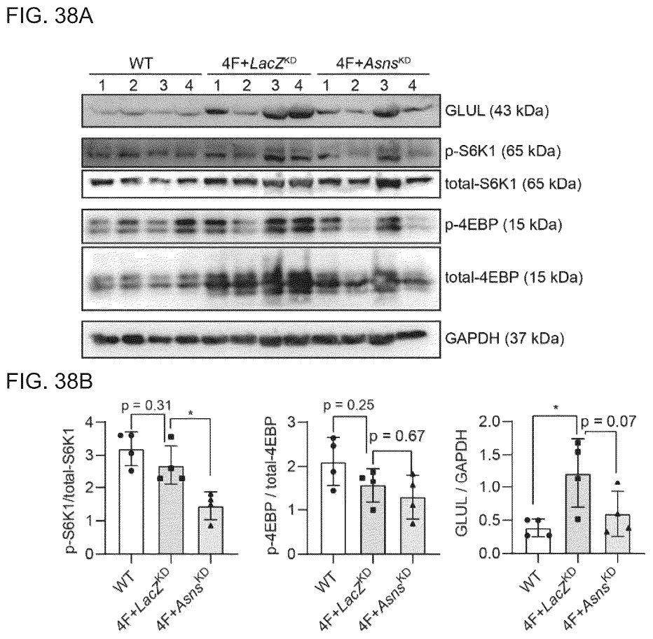

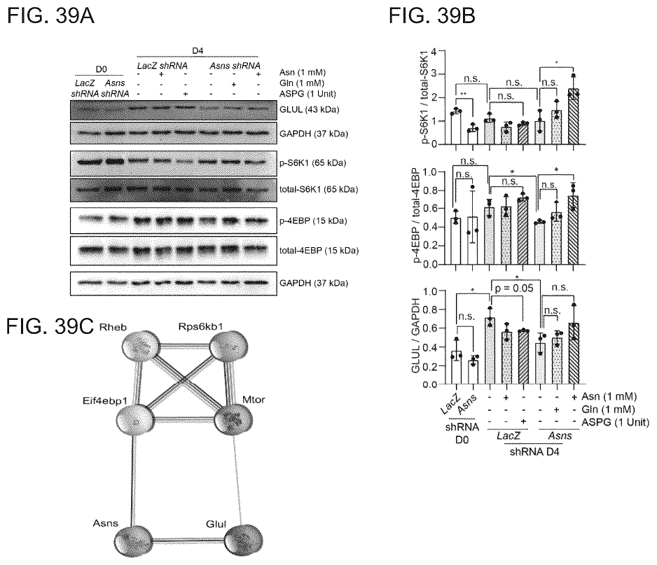

Given that amino acids are known to activate the mTOR Complex 1 (mTORC1) pathway, crucial for protein synthesis, cell survival and cell proliferation in other contexts, it is hypothesised that Asns regulates CMDD through mTORC1. To test this, Western blot was performed to assess total—(t-) and phosphorylated (p-) S6K1 and 4EBP1, two well-characterised downstream effectors of mTORC1. Compared to WT hearts, p-S6K1 and p-4EBP1 in OSKM (4F+LacZ^{KD}) hearts were not significantly changed, but Asns^{KD} (4F+Asns^{KD}) inhibited p-S6K1, indicating impaired mTORC1 activity (FIGS. 38A-38B). In cultured ACMs, again while p-S6K1 and p-4EBP1 were not altered in LacZ^{KD} ACMs during D0 to D4 CMDD, p-4EBP1 was compromised in D4 Asns^{KD}, and restored by Asn treatment. Interestingly, compared to LacZ^{KD} ACMs, p-S6K1 was inhibited in Asns^{KD} ACMs on D0, but not on D4. Even so, Asn promoted p-S6K1 in D4 Asns^{KD} ACMs (FIGS. 39A-39B). From these findings, we conclude that Asns is necessary to maintain mTORC1 pathways during CMDD in both models.

On the other hand, because Asn has been reported to induce glutamine synthetase (GLUL) accumulation in cancer cells under Gln starvation, GLUL expression during CMDD was also assessed. GLUL was significantly upregulated in both cultured ACMs and 4F+LacZ^{KD} hearts, and significantly suppressed by Asns^{KD} (FIGS. 38A-38B and FIG. 39A). This may explain concurrent Gln increase, together with Asn in dedifferentiating CM, even in the presence of Asns upregulation; and also implying an overall requirement led first by Asn for CMDD. Indeed, a recent study implicated Gln-primed mTORC1 activity as being essential for zebrafish heart regeneration (See Miklas et al., 2022). Analysis by STRING also depicts an interaction network between ASNS, GLUL and mTORC1-related proteins (FIG. 39C). To summarise, during CMDD, upregulated Asns, promote Asn and Gln accumulation, and is directly or indirectly required for maintaining CM survival (in cultured ACMs) and progressive CM cell cycle (in OSKM-CMs) through mTORC1 activity.

### Example 10

### Asns Deficiency Inhibited Cardiac Regeneration in the Neonatal Mouse Myocardial Infarction (MI) Model

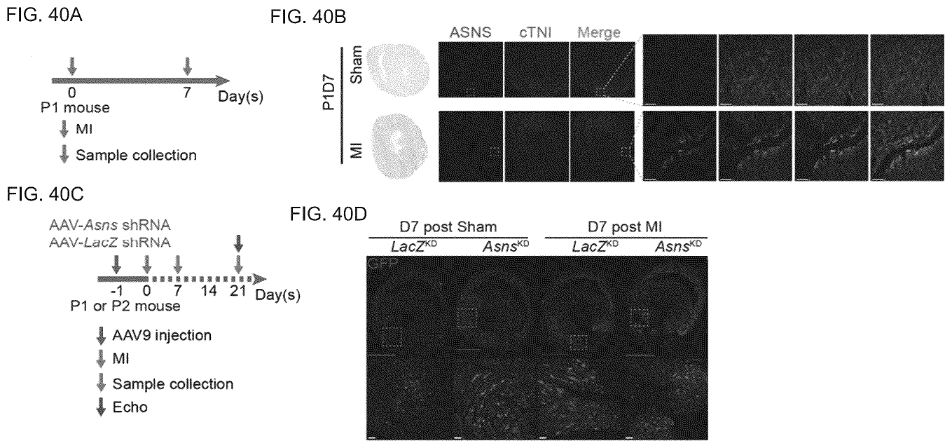

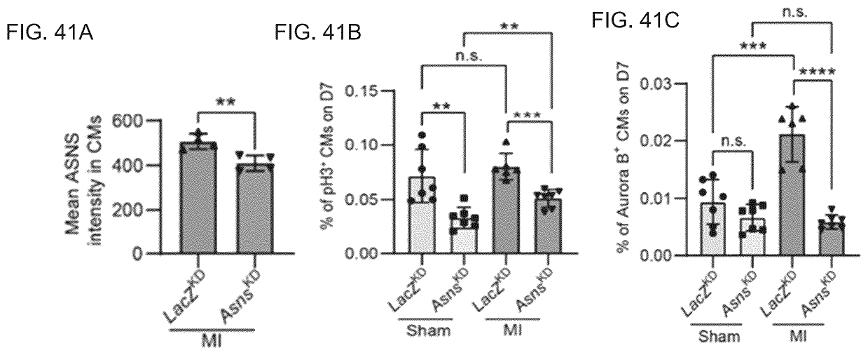

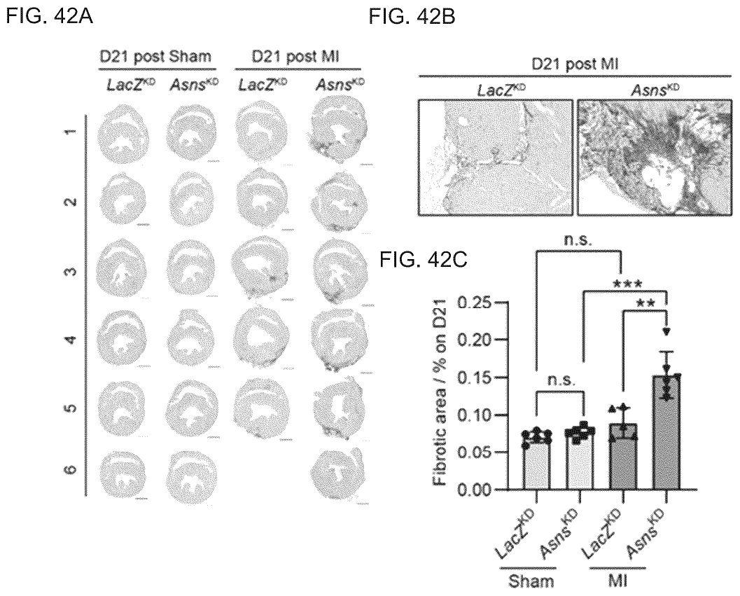

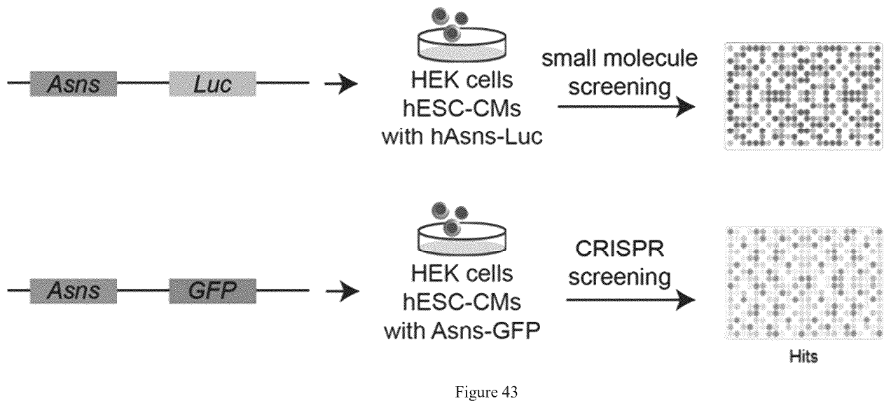

To further verify the commonality as to whether Asns also plays a role in another setting of CMDD, we sought the use of neonatal mouse MI model where CMs undergo CMDD and robust regeneration upon MI in postnatal day 1 (P1) old mice (FIG. 40A). We performed IF staining of P1 mouse hearts 7 days (D7) post MI or a Sham surgery. Clear regional ASNS expression was observed at the MI border zone, compared to remote zones and Sham hearts (FIG. 40B). This is also consistent with published data which we re-analysed, showing increased Asns+ CMs after MI in neonatal mice assessed by single cell RNAseq (Cui M et al., Dev Cell, 2020). More importantly, to fully understand if Asns is required for neonatal mouse heart regeneration, we again injected either AAV-cTnT-Asns shRNA-GFP or AAV-cTnT-LacZ shRNA-GFP into P1 mice, followed by MI/Sham, and harvested tissues at D7 and D21 post-surgery, respectively (FIG. 40C). We used the GFP reporter tag to confirm equal shRNA construct expression in heart samples on D7 (FIG. 40D). ASNS protein expression, especially at the MI border zone (BZ), was significantly suppressed in MI+Asns^{KD} group on D7 (FIG. 41A). Quantification of the mitosis marker pH3+ on D7 showed decrease in Asns^{KD} both after Sham and MI (FIG. 41B), Quantification of Aurora kinase B proved increased CM cytokinesis after MI, which was also supressed by Asns^{KD} (FIG. 41C). Coherent to this, MI+Asns^{KD} group showed more fibrotic scarring than MI+LacZ^{KD} group on D21, and reduced regeneration (FIGS. 42A-42C). In summary, we conclude that Asns is essential for neonatal mouse heart regeneration after MI.

### Example 11

### Screening for ASNS Regulators

ASNS regulators such as inhibitors and activators were screened. Specifically, lentivirus comprising luciferase or GFP reporter driven by human Asns promoter (SEQ ID NO: 2) were transduced into HEK cells and human embryonic stem cells (hESCs), which were then differentiated into cardiomyocytes (hESC-CMs). A library of small molecules was contacted with HEK cells and hESC-CMs with hAsns-Luc, and the signals were detected and analysed to screen for small molecular inhibitors and activators of Asns. In addition, HEK cells and hESC-CMs with hAsns-GFP were used for screening gene regulators of Asns. The HEK cells with hAsns-Luc were seeded in 384-well plate for high throughput screening. The cells were treated with a library of more than 3,000 compounds for 48 hours, followed by luciferase assay and cell viability assay using ONE-Glo™ kit. The readout is the luciferase intensity normalised to cell viability.

### SUMMARY

Adult mammalian cardiomyocytes (CMs) have limited proliferative capacity, but in specifically induced contexts they traverse through cell-cycle re-entry, offering the potential for heart regeneration. Endogenous CM proliferation is preceded by CM dedifferentiation (CMDD), wherein adult CMs revert to a less-matured state that is distinct from the classical myocardial fetal stress gene response associated with heart failure. Yet, very little is known about CMDD as a defined CM cell state in transition. Here, we leveraged two models of in vitro cultured adult mouse CMs and in vivo AAV9 CM-targeted delivery of reprogramming factors (OSKM) in adult mouse to study CMDD. We profiled their transcriptomes using RNA sequencing, in combination with multiple published datasets, with the aim of identifying a common denominator for tracking CMDD.

Next, RNA sequencing and integrated analysis identified Asparagine Synthetase (Asns) as a unique molecular marker gene well correlated with CMDD, required for increased asparagine and for distinct fluxes in other amino acids. While Asns overexpression in OSKM-CMs augmented hallmarks of CMDD, Asns deficiency led to defective regeneration in the neonatal mouse myocardial infarction model, increased cell death of cultured adult CMs and reduced cell cycle in OSKM-CMs, at least in part through disrupting the mTORC1 pathway.

In summary, we have discovered a novel gene, Asns, as both a molecular marker and an essential mediator, marking a distinct threshold that appears in common for at least four models of CMDD, and revealing an Asns/mTORC1 axis dependency for dedifferentiating CMs. Further study will be needed to extrapolate and assess its relevance also to other cell state transitions in heart regeneration.

While the subject matter of this disclosure has been described and shown in considerable detail with reference to certain illustrative aspects, including various combinations and sub-combinations of features, those skilled in the art will readily appreciate other aspects and variations and modifications thereof as encompassed within the scope of the present disclosure. Moreover, the descriptions of such aspects, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims.

### BIBLIOGRAPHY

1. Haenebalcke L, Goossens S, Dierickx P, Bartunkova S, D'Hont J, Haigh K, Hochepied 762 T, Wirth D, Nagy A, Haigh J J. The ROSA26-iPSC Mouse: A Conditional, Inducible, 763 and Exchangeable Resource for Studying Cellular (De) Differentiation. Cell Rep 764 [Internet]. 2013; 3:335-341.
2. Ackers-Johnson M, Li P Y, Holmes A P, O'Brien S M, Pavlovic D, Foo R S. A Simplified, Langendorff-Free Method for Concomitant Isolation of Viable Cardiac Myocytes and Nonmyocytes from the Adult Mouse Heart. Circ Res. 2016; 119:909-920.
3. Kabaeva Z, Zhao M, Michele D E. Blebbistatin extends culture life of adult mouse cardiac myocytes and allows efficient and stable transgene expression. Am J Physiol—Hear Circ Physiol. 2008; 294:1667-1674.
4. Wakimoto H, Seidman J G, Foo R S Y, Jiang J. AAV9 delivery of shRNA to the mouse 525 heart. Curr Protoc Mol Biol. 2016; 2016:1-9.
5. Aharonov A, Shakked A, Umansky K B, Savidor A, Genzelinakh A, Kain D, Lendengolts D, Revach O Y, Morikawa Y, Dong J, Levin Y, Geiger B, Martin J F, Tzahor E. ERBB2 drives YAP activation and EMT-like processes during cardiac regeneration. Nat Cell Biol [Internet]. 2020; 22:1346-1356.
6. Ikeda S, Mizushima W, Sciarretta S, Abdellatif M, Zhai P, Mukai R, Fefelova N, Oka S I, Nakamura M, Del Re D P, Farrance I, Park J Y, Tian B, Xie L H, Kumar M, Hsu C P, Sadayappan S, Shimokawa H, Lim D S, Sadoshima J. Hippo Deficiency Leads to Cardiac Dysfunction Accompanied by Cardiomyocyte Dedifferentiation during Pressure Overload. Circ Res. 2019; 124:292-305.
7. Leach J P, Heallen T, Zhang M, Rahmani M, Morikawa Y, Hill M C, Segura A, Willerson J T, Martin J F. Hippo pathway deficiency reverses systolic heart failure after infarction. Nature [Internet]. 2017; 550:260-264.
8. O'Meara C C, Wamstad J A, Gladstone R A, Fomovsky G M, Butty V L, Shrikumar A, Gannon J B, Boyer L A, Lee R T. Transcriptional reversion of cardiac myocyte fate during mammalian cardiac regeneration. Circ Res. 2015; 116:804-815.
9. Cui M, Wang Z, Chen K, Shah A M, Tan W, Duan L, Sanchez-Ortiz E, Li H, Xu L, Liu N, Bassel-Duby R, Olson E N. Dynamic Transcriptional Responses to Injury of Regenerative and Non-regenerative Cardiomyocytes Revealed by Single-Nucleus RNA Sequencing. Dev Cell [Internet]. 2020; 53:102-116.e8.
10. Quaife-Ryan G A, Sim C B, Ziemann M, Kaspi A, Rafehi H, Ramialison M, El-Osta A, Hudson J E, Porrello E R. Multicellular Transcriptional Analysis of Mammalian Heart Regeneration. Circulation. 2017; 136:1123-1139.
11. Mohamed T M A, Ang Y S, Radzinsky E, Zhou P, Huang Y, Elfenbein A, Foley A, Magnitsky S, Srivastava D. Regulation of Cell Cycle to Stimulate Adult Cardiomyocyte Proliferation and Cardiac Regeneration. Cell [Internet]. 2018; 173:104-116.e12.
12. Ben-Yair R, Butty V L, Busby M, Qiu Y, Levine S S, Goren A, Boyer L A, Geoffrey Burns C, Burns C E. H3K27me3-mediated silencing of structural genes is required for zebrafish heart regeneration. Dev. 2019; 146.
13. Ye L, D'Agostino G, Loo S J, Wang C X, Su L P, Tan S H, Tee G Z, Pua C J, Pena E M, Cheng R B, Chen W C, Abdurrachim D, Lalic J, Tan R S, Lee T H, Zhang J, Cook S A. Early Regenerative Capacity in the Porcine Heart. Circulation. 2018; 138:2798-2808.
14. Schachterle W, Rojas A, Xu S M, Black B L. ETS-dependent regulation of a distal Gata4 cardiac enhancer. Dev Biol. 2012; 361:439-449.
15. Tskhovrebova L, Trinick J. Making muscle elastic: The structural basis of myomesin stretching. PLOS Biol. 2012; 10.
16. Shimshek D R, Kim J, Hübner MR, Spergel D J, Buchholz F, Casanova E, Stewart A F, Seeburg P H, Sprengel R. Codon-improved Cre recombinase (iCre) expression in the mouse. Genesis. 2002; 32:19-26.
17. Muzumdar M D, Tasic B, Miyamichi K, Li L, Luo L. A global double-fluorescent Cre reporter mouse. Genesis. 2007; 45:593-605.
18. Chen Y, Lüttmann F F, Schoger E, Schöler HR, Zelarayan L C, Kim K-P, Haigh J J, Kim J, Braun T. Reversible reprogramming of cardiomyocytes to a fetal state drives heart regeneration in mice. Science. 2021; 373:1537-1540.
19. Kubin T, Poling J, Kostin S, Gajawada P, Hein S, Rees W, Wietelmann A, Tanaka M, Lörchner H, Schimanski S, Szibor M, Warnecke H, Braun T. Oncostatin M is a major mediator of cardiomyocyte dedifferentiation and remodeling. Cell Stem Cell. 2011; 9:420-432.
20. Miklas J W, Levy S, Hofsteen P, Mex D I, Clark E, Muster J, Robitaille A M, Sivaram G, Abell L, Goodson J M, Pranoto I, Madan A, Chin M T, Tian R, Murry C E, Moon R T, Wang Y, Ruohola-Baker H. Amino acid primed mTOR activity is essential for heart regeneration. iScience [Internet]. 2022; 25:103574.
21. Morikawa Y, Zhang M, Heallen T, Leach J, Tao G, Xiao Y, et al. Actin cytoskeletal remodeling with protrusion formation is essential for heart regeneration in Hippo-deficient mice. Sci Signal. 2015; 8 (375).
22. D'Uva G, Aharonov A, Lauriola M, Kain D, Yahalom-Ronen Y, Carvalho S, et al. ERBB2 triggers mammalian heart regeneration by promoting cardiomyocyte dedifferentiation and proliferation. Nat Cell Biol. 2015; 17 (5): 627-38.

1. A method of detecting cardiomyocyte dedifferentiation (CMDD) in a test sample from a subject, comprising:
(a) determining an expression level of an Asparagine Synthetase (Asns) gene, protein or mRNA in the test sample;
(b) comparing the expression level of the Asns gene, protein or mRNA in the test sample to that in a reference sample,
wherein a difference in the level of the Asns gene, protein or mRNA in the test sample compared to the reference sample indicates that the test sample contains dedifferentiating cardiomyocytes.

2. The method according to claim 1, wherein the test sample comprises isolated cardiomyocytes from cardiac tissue or from a biopsy sample.

3. The method according to claim 1, wherein the CMDD is in the progression to achieve cardiac regeneration.

4. The method according to claim 1, wherein the reference sample is obtained from a healthy heart that does not show CMDD.

5. The method according to claim 1,
wherein determining the expression level of the Asns gene, protein or mRNA comprises using:
i) an antibody which specifically binds to Asns protein;
ii) an Asns-specific oligonucleotide primer;
iii) an Asns-specific oligonucleotide probe; or
iv) an expression vector comprising an Asns gene promoter operably linked to a detectable heterologous reporter gene.

6. The method according to claim 5, wherein the Asns gene promoter is a mouse or human Asns gene promoter.

7. The method according to claim 6, wherein the mouse Asns gene promoter comprises a polynucleic acid sequence having at least 85% sequence identity with the polynucleic acid sequence set forth in SEQ ID NO: 1 and the human Asns gene promoter comprises a polynucleic acid sequence having at least 85% sequence identity with the polynucleic acid sequence set forth in SEQ ID NO: 2.

8. A method of augmenting or suppressing cardiomyocyte dedifferentiation (CMDD), comprising:
(a) contacting a cardiomyocyte with an expression vector comprising a polynucleotide sequence which increases the expression or activity of Asns, wherein the increase in expression or activity of Asns augments CMDD; or
(b) contacting a cardiomyocyte with an expression vector comprising a polynucleotide sequence which suppresses the expression or activity of Asns, wherein the decrease in expression or activity of Asns suppresses CMDD.

9. The method according to claim 8, wherein the polynucleotide sequence which increases the expression or activity of Asns encodes an Asns protein.

10. The method according to claim 9, wherein the Asns protein comprises the amino acid sequence set forth in SEQ ID NO: 3 or SEQ ID NO: 4.

11. The method according to claim 8, wherein the polynucleotide sequence which increases the expression or activity of Asns is operably linked to a cardiomyocyte-specific promoter.

12. The method according to claim 8, wherein said augmenting cardiomyocyte dedifferentiation is to enhance cardiac regeneration.

13. (canceled)

14. The method of claim 8, wherein said suppressing cardiomyocyte dedifferentiation is to control cardiac regeneration.

15. The method according to claim 8, wherein the polynucleotide sequence which suppresses the expression or activity of Asns comprises shRNA or siRNA targeting Asns.

16.-24. (canceled)

25. A method of screening for a therapeutic or prophylactic drug having cardiomyocyte dedifferentiation-promoting activity, comprising:
(a) contacting a candidate therapeutic or prophylactic drug with cardiomyocytes or HEK293T cells;
(b) detecting the expression level of Asns protein, mRNA, or Asns gene in the presence of the candidate drug; and
(c) Identifying the candidate therapeutic or prophylactic drug as having cardiomyocyte dedifferentiation- or proliferation-promoting activity if the expression level of the Asns protein, mRNA or Asns gene increases compared to that of control cardiomyocytes or HEK293T cells unexposed to the candidate therapeutic or prophylactic drug.

26. The method of claim 25, wherein before the contacting step, the cardiomyocytes are transformed with a vector comprising a polynucleotide sequence comprising a heterologous Asns gene promoter operably linked to a reporter gene.

27. The method of claim 25, which is performed in vitro.

28. A method of treatment of heart failure, comprising administering to a subject in need of such treatment a combination of an efficacious amount of:
a) an expression vector comprising a polynucleotide sequence encoding Asns, and
b) (i) an inhibitor of Hippo-Yap signalling or
(ii) an activator of the ErbB2 pathway, or
(iii) an upregulator of the expression of OSKM factors Oct4, Sox2, Klf4 and Myc, wherein the method promotes regeneration of heart tissue greater than that induced in the absence of the Asns expression vector.

29. The method according to claim 28, wherein the polynucleotide sequence encoding Asns is operably linked to an Asns promoter of SEQ ID NO: 1 or SEQ ID NO: 2.

30. The method according to claim 28, wherein the expression vector comprises a virus selected from the group comprising adenovirus, adeno-associated virus and lentivirus.

31.-38. (canceled)


SEQUENCE LISTING (7 sequences)

SEQ ID NO: 1 - DNA, 1192 bp, Mus musculus
  source 1..1192: mol_type = genomic DNA
ttaggacaaacatttctttttattgtacaatgaatttgtaggcactattggttcaagagtgcttttaatctctgcagggagattacctggggttcttcttagctgctcagtagtagtagttatttgtgtgtgtgtgtgtctgtgtgtggtgtccttctaggtattcctatgttacttatgtatccacatctatatatcagacactctgttaggaagtactgaagactacaattaaggagtaggagatctattgctgatgggacactggacctggcggcccgacctctgggcttatcctgcctgcagtgtcacagcaccttaaggaacagaagcagaacacctcctggctctcggccccaggatgcacgtttgccttggcctccgccccgcctggcgtcgctcgtcagcactgcgggccacgcccctgcgcacgggttgggtcccagccacatgatgaaacttcccgcacgcgttacagaagcccggcgactctaaggtgggaagcggcctccaaccggtcttgtcactgcgctgcctctgctccaccttctctggccctggccgctagtgctcaggtaagcgggagccgggtgctcagcgcggagcgctctgctcgcctgggaggccggaggatgtgctgtccgcgcgggcacatgtcccgggagttcggatgcggagcgctcgggtccggcgccagaccgatgctgcggtgcagcgcgaggcagggcggggcgggcgcggggcgattgttgcgcacctaggacggagctgccaggaaagctttcatgtgagcgaagtgcagcgcggaggcagggccgctccagacagaaggcgaaggtggccttcatttagacaccataaatatttttttttactgtatgacgtctagatctgggaaatttcagccatccgaaggttttgcctgggactaggtggacgcttgcacaagaccagctgtaattgggagccccaggctagtgtaggggtctcgctctccagcaggacagaggctctaccaagctctagagcgagtgtctctgggtctgtctggatccggatttctgagcactcactcacctcgctgctcaggcccttcctccactccagttccatactcgcagccacgccgggaaaaatctgtttaaggagacaggcaaccaagtacgggaaaaggcaaaggaattggcgtgct

SEQ ID NO: 2 - DNA, 715 bp, Homo sapiens
  source 1..715: mol_type = genomic DNA
ttcttaggacaagcttttctctgcactgcataataaatcagacggcagtgcaacaaaaaaacggtgatgatttcccgaagaacaaaccaagttcagaagcctccctaattgtcagaacagcaggtagcctgggcggagctctgaggctatccttaaacaggcgcactgagacgcaaggcgctggaaacaaaagagctcctccttgcgcccttccgccgccccacttagtcctgctccgccccggacaccccgcggccccgcccctgtgcgcgctggttggtcctcgcaggcatgatgaaacttcccgcacgcgttacaggagccaggtcggtataagcgccagcggcctcgccgcccgtcaagctgtccacatccctggcctcagcccgccacatcaccctgacctgcttacgcccaggtgagccgggtcctgcgccccgtgccctgcgccccgcgccagcatcctcctcgcgtgccctggggctgctccggaaggaaggggtggaggatgcggtcttcagcctggcacccggcagacgcggttcgcgggcgcctgctgatgctgcggctcctgcggtagggagggcggggcggacgcctagcgattgttacgcacctgacagctagcggaaggacctttctgtcctggttccctgaagcatcgagagggaaccgcggccggcaggccggaccgctccggagaggaggcagcggc

SEQ ID NO: 3 - protein, 561 aa, Mus musculus
MCGIWALFGSDDCLSVQCLSAMKIAHRGPDAFRFENVNGYTNCCFGFHRLAVVDPLFGMQPIRVRKYPYLWLCYNGEIYNHKALQQRFEFEYQTNVDGEIILHLYDKGGIEKTICMLDGVFAFILLDTANKKVFLGRDTYGVRPLFKAMTEDGFLAVCSEAKGLVSLKHSTTPFLKVEPFLPGHYEVLDLKPNGKVASVEMVKYHHCTDEPLHAIYDSVEKLFPGFDLETVKNNLRILFDNAIKKRLMTDRRIGCLLSGGLDSSLVAASLLKQLKEAQVQYPLQTFAIGMEDSPDLLAARKVANYIGSEHHEVLFNSEEGIQALDEVIFSLETYDITTVRASVGMYLISKYIRKNTDSVVIFSGEGSDELTQGYIYFHKAPSPEKAEEESERLLKELYLFDVLRADRTTAAHGLELRVPFLDHRFSSYYLSLPPDMRIPKNGIEKHLLRETFEDCNLLPKEILWRPKEAFSDGITSVKNSWFKILQDYVEHQVDDEMMSAASQKFPFNTPKTKEGYFYRQIFERHYPGRADWLTHYWMPKWINATDPSARTLTHYKSAAKA

SEQ ID NO: 4 - protein, 540 aa, Homo sapiens
MKIAHRGPDAFRFENVNGYTNCCFGFHRLAVVDPLFGMQPIRVKKYPYLWLCYNGEIYNHKKMQQHFEFEYQTKVDGEIILHLYDKGGIEQTICMLDGVFAFVLLDTANKKVFLGRDTYGVRPLFKAMTEDGFLAVCSEAKGLVTLKHSATPFLKVEPFLPGHYEVLDLKPNGKVASVEMVKYHHCRDVPLHALYDNVEKLFPGFEIETVKNNLRILFNNAVKKRLMTDRRIGCLLSGGLDSSLVAATLLKQLKEAQVQYPLQTFAIGMEDSPDLLAARKVADHIGSEHYEVLFNSEEGIQALDEVIFSLETYDITTVRASVGMYLISKYIRKNTDSVVIFSGEGSDELTQGYIYFHKAPSPEKAEEESERLLRELYLFDVLRADRTTAAHGLELRVPFLDHRFSSYYLSLPPEMRIPKNGIEKHLLRETFEDSNLIPKEILWRPKEAFSDGITSVKNSWFKILQEYVEHQVDDAMMANAAQKFPFNTPKTKEGYYYRQVFERHYPGRADWLSHYWMPKWINATDPSARTLTHYKSAVKA

SEQ ID NO: 5 - RNA, 22 nt, synthetic construct
  source 1..22: mol_type = other RNA
gtctcaaagcgaaatgcatctg

SEQ ID NO: 6 - RNA, 21 nt, synthetic construct
  source 1..21: mol_type = other RNA
ctttagcgactaaacacatca

SEQ ID NO: 7 - DNA, 1686 bp, Mus musculus
  source 1..1686: mol_type = genomic DNA
atgtgtggcatctgggccctcttcggcagcgatgactgcctttccgtgcagtgtctgagtgcgatgaagatcgcgcacagggggccagatgcatttcgctttgagaatgtcaatggatacaccaactgctgctttggctttcaccgcttggctgtggttgacccgctgtttggaatgcagccgataagagtgaggaaatacccttatttgtggctctgttacaatggtgaaatctacaaccacaaggcgctacagcaacgttttgaatttgaatatcagaccaatgtggatggtgagattatcctccacctctatgacaaaggaggcatcgagaaaaccatctgtatgctggacggggtgtttgcattcatcttactggacactgccaataagaaagtatttctgggcagagacacctatggagtcaggcccttgtttaaagccatgacagaagatgggtttctggctgtgtgttcagaagctaaaggccttgtttccttaaaacactccaccactcccttcttaaaagtggagcccttccttcctggacactatgaagttttggatttaaaaccaaatggcaaagttgcgtctgtggaaatggtcaaataccatcactgtacggatgaaccattgcatgccatctatgacagcgtggagaaactcttcccaggctttgacctagagaccgtgaagaacaatctgcgtatcctttttgacaacgctatcaagaaacgcttgatgacagaccggaggattggctgccttttatcagggggcctggactcgagcttggttgctgcctctctgctgaagcaactcaaggaggcccaagttcagtatcctctccagacatttgctattggcatggaggacagccccgatctcctggccgctagaaaggtggcaaattatattggaagcgagcatcatgaagtcctttttaactctgaagaaggcattcaggccctggatgaagtcatattttccttggaaacttatgatattacgacagttcgggcatctgtgggcatgtatttaatttccaagtatattcggaagaacacagacagcgtggtgatcttctccggagaggggtcagatgaacttacacagggctatatatatttccacaaggctccttcccctgagaaggccgaggaggagagtgagagactgctgaaggaactctacctgtttgatgttctccgggccgaccgcactactgctgcacacggtctcgaactgagagtcccctttctggatcatcggttttcttcctattacctgtctctgccgccagatatgagaattccaaaaaatggcatagaaaaacatctcctgagagagacttttgaggactgcaacctgctacccaaagagattctctggcgacccaaagaagccttcagtgatgggatcacctcagtcaagaactcctggttcaagattttgcaggactatgttgaacatcaggttgatgatgagatgatgtctgcagcctcccagaagtttcccttcaatactcccaaaactaaggaaggctacttctaccgtcagatctttgaacgccattacccaggccgggctgattggctgactcattattggatgcctaagtggatcaatgctactgacccttctgcccgcactctgacccattataagtcagctgccaaagcttag